(19) United States
(12) Patent Application Publication
(10) Pub. No.: US 2005/0080096 A1
(43) Pub. Date: Apr. 14, 2005

(54) Condensed heterocyclic compounds

(76) Inventors: Junya Ishida, Osaka (JP); Kouji Hattori, Osaka (JP); Yoshiyuki Kido, Osaka (JP); Hirofumi Yamamoto, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/501,334

(22) PCT Filed: Jan. 27, 2003
(86) PCT No.: PCT/JP03/00708

(30) Foreign Application Priority Data
Jan. 29, 2002 (AU) PS 0197

(51) Int. Cl. A61K 31/519; A61K 31/517; A61K 31/503; A61K 31/4745; A61K 31/47

(52) U.S. Cl. 514/260.1; 514/264.1; 514/248; 514/266.3; 514/309; 544/236; 544/279; 544/280; 544/286; 546/122; 546/141; 514/300

(57) ABSTRACT

A condensed heterocyclic compound having poly(adenosine 5′-diphospho-ribose)polymerase (PARP) inhibitory activity by the formula (I): wherein R¹ is hydrogen, halogen, lower alkyl or lower alkoxy, A and two adjacent carbon atoms of the six membered ring to be bonded with A form benzene ring, pyridine ring, etc, —Y¹═Y²— is formula (II) wherein L¹¹, L¹², L¹³ and L¹⁴ is (1) lower alkylene, (2) lower alkenylene, etc, and R²¹, R²², R²³ and R²⁴ is (1) cyclic amino group, which is substituted with phenyl optionally substituted with one or more suitable substituent(s), etc. provided that when A and two adjacent carbon atoms of the six membered ring to be bonded with A form benzene ring, then —Y¹═Y²— is formula (III) or its prodrug, or their salts.

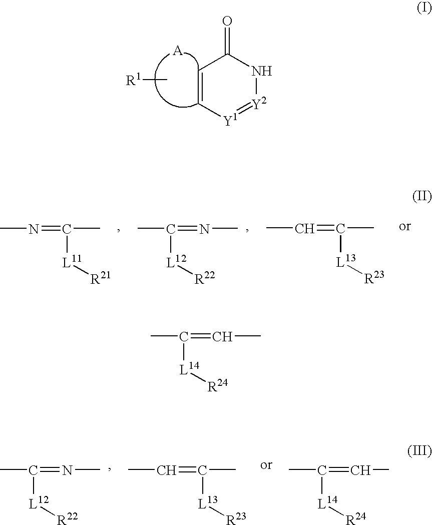

### TECHNICAL FIELD

This invention relates to novel condensed heterocyclic compounds having pharmacological activity, to a process for their production and to a pharmaceutical composition containing the same.

### BACKGROUND ART

Poly (adenosine 5′-diphospho-ribose) polymerase [“poly (ADP-ribose) polymerase” or “PARP”, which is also sometimes called “PARS” for “poly (ADP-ribose) synthetase”] is an enzyme located in the nuclei of cells of various organs, including muscle, heart and brain cells. PARP plays a physiological role in the repair of strand breaks in DNA. Once activated by damaged DNA fragments, PARP catalyzes the attachment of up to 100 ADP-ribose units to a variety of nuclear proteins, including histones and PARP itself.

Some condensed heterocyclic compound having inhibitory activity of PARP have been known, for example, in WO95/24379, WO98/33802 and WO99/11624.

### DISCLOSURE OF THE INVENTION

This invention relates to novel condensed heterocyclic compound, which have pharmaceutical activity such as PARP inhibiting activity, to a process for their production, to a pharmaceutical composition containing the same and to a use thereof.

One object of this invention is to provide the novel condensed heterocyclic compound, which have a PARP inhibiting activity.

Another object of this invention is to provide a process for production of the condensed heterocyclic compound.

A further object of this invention is to provide a pharmaceutical composition containing the condensed heterocyclic compound as an active ingredient.

Still further object of this invention is to provide a use of the condensed heterocyclic compound for manufacturing a medicament for treating or preventing various diseases, or a method of treating or preventing various diseases by administering the condensed heterocyclic compound in an effective amount to inhibit PARP activity.

Thus, the present invention provides the following.

[1] A compound of the formula (I): 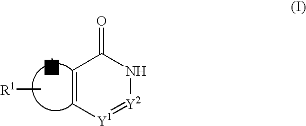
wherein
R¹ is hydrogen, halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from the group consisting of nitrogen atom, oxygen atom, and sulfur atom, —Y¹═Y²— is 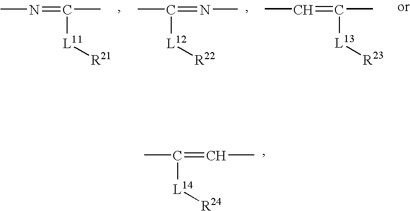
[wherein L¹¹, L¹², L¹³ and L¹⁴ is
   (1) lower alkylene,
   (2) lower alkenylene,
   (3) cyclo(lower)alkylene,
   (4) cyclo(lower)alkenylene,
   (5) diradical of saturated- or unsaturated monocyclic group with one or more nitrogen atom(s), which is obtained after removal of one hydrogen atom from said monocyclic group, or
   (6) —N(R³)-L- (wherein R³ is hydrogen or lower alkyl, and L is lower alkylene or lower alkenylene), and
R²¹, R²², R²³ and R²⁴ is
   (1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
   (2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
   (3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.],
provided that
when A and two adjacent carbon atoms of the six membered ring to be bonded with A form benzene ring,
then —Y¹═Y²— is 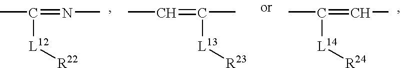
or its prodrug, or their salts.

[2] The compound according to [1], wherein 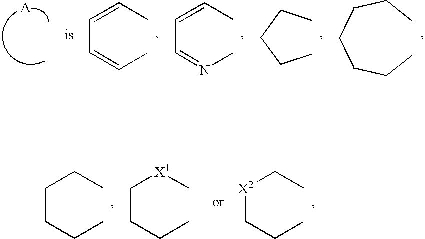
[wherein X¹ and X² is N, O or S].

[3] The compound according to [2], wherein
R¹ is hydrogen, and
R²¹, R²², R²³ and R²⁴ is tetrahydropyridyl, piperidyl or piperazinyl, each of which is substituted with aryl optionally substituted with halogen.

[4] The compound according to any one of [1], [2] and [3], wherein
L is lower alkylene.

[5] The compound according to any one of [1], [2], [3] and [4], wherein
—Y¹═Y²— is

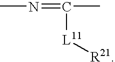

[6] A pharmaceutically composition comprising a compound of the formula (I): 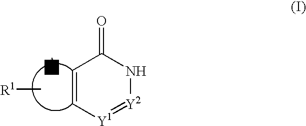
wherein
R¹ is halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from the group consisting of nitrogen atom, oxygen atom, and sulfur atom,
—Y¹═Y²— is 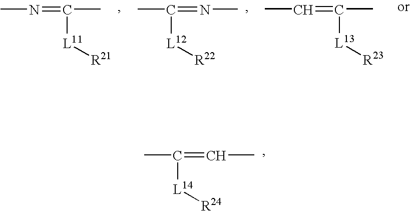
[wherein L¹¹, L¹², L¹³ and L¹⁴ is
   (1) lower alkylene,
   (2) lower alkenylene,
   (3) cyclo(lower)alkylene,
   (4) cyclo(lower)alkenylene,
   (5) diradical of saturated- or unsaturated monocyclic group with one or more nitrogen atom(s), which is obtained after removal of one hydrogen atom from said monocyclic group, or
   (6) —N(R³)-L- (wherein R³ is hydrogen or lower alkyl, and L is lower alkylene or lower alkenylene), and
R²¹, R²², R²³ and R²⁴ is
   (1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
   (2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
   (3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.],
provided that
when A and two adjacent carbon atoms of the six membered ring to be bonded with A form benzene ring,
then —Y¹═Y²is 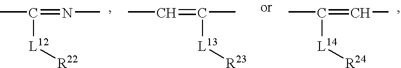
or its prodrug, or their pharmaceutically acceptable salts, and a pharmaceutically acceptable carrier, wherein said compound is present in an amount effective for inhibiting PARP activity.

[7] The pharmaceutical composition of [6] for treating or preventing diseases ascribed by NMDA- and NO-induced toxicity.

[8] The pharmaceutical composition of [6] for extending the lifespan or proliferative capacity of cells or altering gene expression of senescent cells

[9] The pharmaceutical composition of [6] for treating or preventing tissue damage resulting from cell damage or death due to necrosis or apoptosis; neural tissue damage resulting from ischernia and reperfusion injury, neurological disorders and neurodegenerative diseases; neurodegenerative diseases; head trauma; stroke; Alzheimer's disease; Parkinson's disease; epilepsy; Amyotrophic Lateral Scleosis (ALS); Huntington's disease; schizophrenia; chronic pain; ischemia and nloss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; arteriosclerosis; osteoarthritis; osteoporosis; muscular dystrophy; degenerative diseases of skeletal muscle involving replicative senescence; age-related macular degeneration; immune senescence; AIDS; other immune senescence diseases; inflammatory bowel disorders (e.g., colitis); arthritis; diabetes; endotoxic shock; septic shock; or tumor.

[10]A method of inhibiting PARP activity comprising administering a compound of the formula: 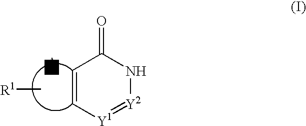
wherein
R¹ is hydrogen, halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from the group consisting of nitrogen atom, oxygen atom, and sulfur atom,
—Y¹═Y²— is 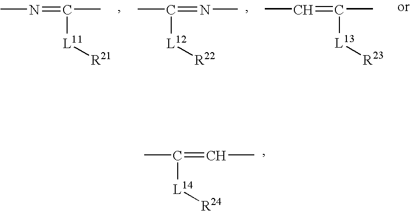
[wherein L¹¹, L¹², L¹³ and L¹⁴ is
   (1) lower alkylene,
   (2) lower alkenylene,
   (3) cyclo(lower)alkylene,
   (4) cyclo(lower)alkenylene,
   (5) diradical of saturated- or unsaturated monocyclic group with one or more nitrogen atom(s), which is obtained after removal of one hydrogen atom from said monocyclic group, or
   (6) —N(R³)-L- (wherein R³ is hydrogen or lower alkyl, and L is lower alkylene or lower alkenylene), and
R²¹, R²², R²³ and R²⁴ is
   (1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
   (2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
   (3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.],
provided that
when A and two adjacent carbon atoms of the six membered ring to be bonded with A form benzene ring,
then —Y¹═Y² is 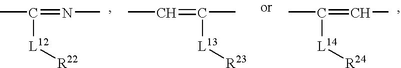
or its prodrug, or their salts.

[11] A use of a compound of the formula (I): 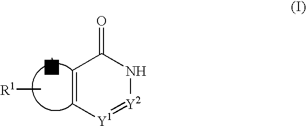
wherein
R¹ is hydrogen, halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from the group consisting of nitrogen atom, oxygen atom, and sulfur atom,
—Y¹═Y²— is 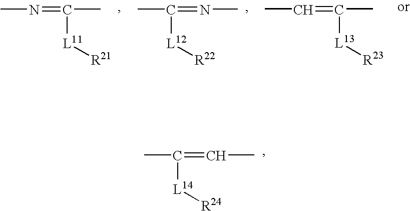
[wherein L¹¹, L¹², L13 and L¹⁴ is
   (1) lower alkylene,
   (2) lower alkenylene,
   (3) cyclo(lower)alkylene,
   (4) cyclo(lower)alkenylene,
   (5) diradical of saturated- or unsaturated monocyclic group with one or more nitrogen atom(s), which is obtained after removal of one hydrogen atom from said monocyclic group, or
   (6) —N(R³)-L- (wherein R³ is hydrogen or lower alkyl, and L is lower alkylene or lower alkenylene), and
R²¹, R²², R²³ land R²⁴ is
   (1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
   (2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
   (3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.],
provided that
when A and two adjacent carbon atoms of the six membered ring to be bonded with A form benzene ring,
then —Y¹═Y²— is 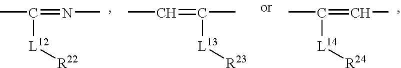
   or its prodrug, or their pharmaceutically acceptable salts, for manufacturing a medicament for inhibiting PARP activity.

The condensed heterocyclic compound of this invention can be represented by the following formula (I): 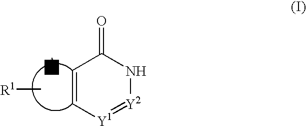
wherein
R¹ is hydrogen, halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from the group consisting of nitrogen atom, oxygen atom, and sulfur atom,
—Y¹═Y²— is 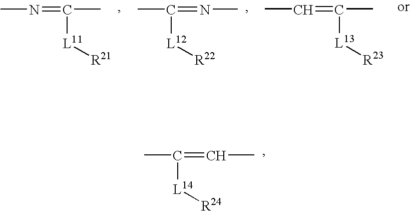
[wherein L¹¹, L¹², L¹³ and L¹⁴ is
   (1) lower alkylene,
   (2) lower alkenylene,
   (3) cyclo(lower)alkylene,
   (4) cyclo(lower)alkenylene,
   (5) diradical of saturated- or unsaturated monocyclic group with one or more nitrogen atom(s), which is obtained after removal of one hydrogen atom from said monocyclic group, or
   (6) —N(R³)-L- (wherein R³ is hydrogen or lower alkyl, and L is lower alkylene or lower alkenylene), and
R²¹, RR3 and R2⁴ is
   (1) cyclic amino group, which is substituted with phenyl optionally substituted with one or more suitable substituent(s) selected from the group consisting of halogen, nitro, lower alkoxy, lower alkyl, halo(oower)alkyl, halo(lower)alkoxy and phenyl, and which is optionally substituted with lower alkyl,
   (2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
   (3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.],
provided that
when A and two adjacent carbon atoms of the six membered ring to be bonded with A form benzene ring,
then —Y¹═Y²— is 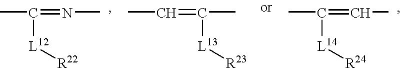
   or its prodrug, or their salts.

The compound (I) or its prodrug, or their salt can be prepared by the following processes. In the following formulae, compounds may be prodrugs or their salts. 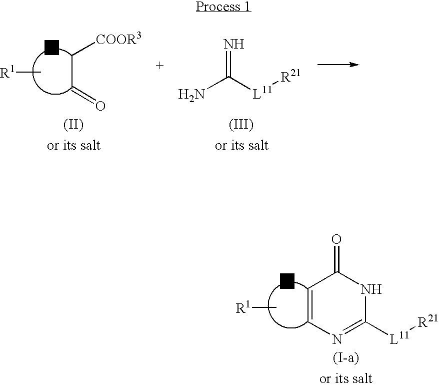
[wherein, R¹, R²l and A are each as defined above, and R³ is lower alkyl.]

In this process, the compound (I-a) or its salts can be produced by reacting the compound (II) or its salt and compound (III) in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as an alcohol (e.g., methanol, ethanol or isopropyl alcohol), ether (e.g., tetrahydrofuran, dioxane, diethylether), amide (e.g., N, N-dimethylformamide, N, N-dimethylacetamide), nitrile (e.g., acetonitrile), or any other organic solvent which does not adversely affect the reaction. The reaction may be usually carried out under cooling to heating since the reaction temperature is not critical. 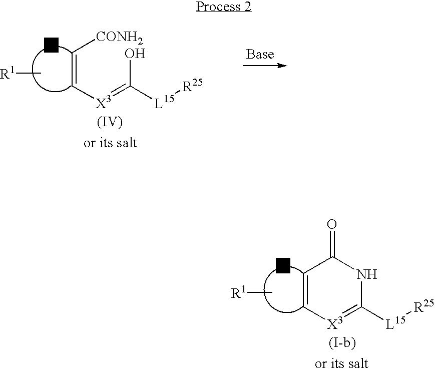
[wherein, R¹ and A are each as defined above, and X³ is CH or N, L¹⁵ has a same meaning of L¹¹ or L¹³, and R²⁵ has a same meaning of R²¹ or R²³.]

In this process, the compound (I-b) can be produced by subjecting the compound (IV) to cyclization reaction in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as water, an alcohol (e.g., methanol, ethanol or isopropyl alcohol), ether (e.g., tetrahydrofuran, dioxane, diethylether), amide (e.g., N, N-dimethylformamide, N, N-dimethylacetamide), nitrile (e.g., acetonitrile), or any other organic solvent which does not adversely affect the reaction. The reaction may be usually carried out under cooling to heating since the reaction temperature is not critical. 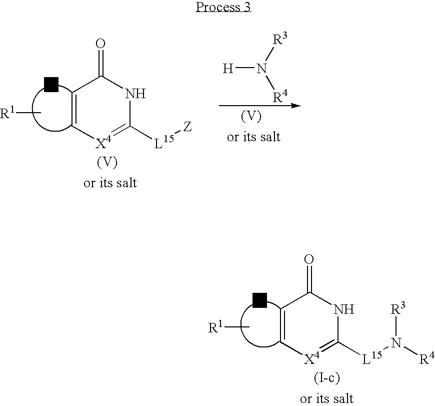
[wherein, R¹ and A are each as defined above, and X⁴ is CH or N, L¹⁵ has a same meaning of L¹¹ or L¹³, Z is halogen, and 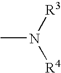
is substituted cyclic amino groups or optionally substituted amino group.]

In this process, the compound (I-c) or its salts can be produced by reacting the compound (IV) or its salt and compound (V) in the presence of base, such as inorganic bases, for example, an alkali metal [e.g., sodium or potassium], alkoxide, hydroxide, carbonate or bicarbonate thereof, or organic bases such as a trialkylamine [e.g., trimethylamine or triethylamine] or the like.

The reaction is usually carried out in a conventional solvent such as an alcohol (e.g., methanol, ethanol or isopropyl alcohol), ether (e.g., tetrahydrofuran, dioxane, diethylether), amide (e.g., N, N-dimethylformamide, N, N-dimethylacetamide), nitrile (e.g., acetonitrile), or any other organic solvent which does not adversely affect the reaction. The reaction may be usually carried out under cooling to heating since the reaction temperature is not critical. 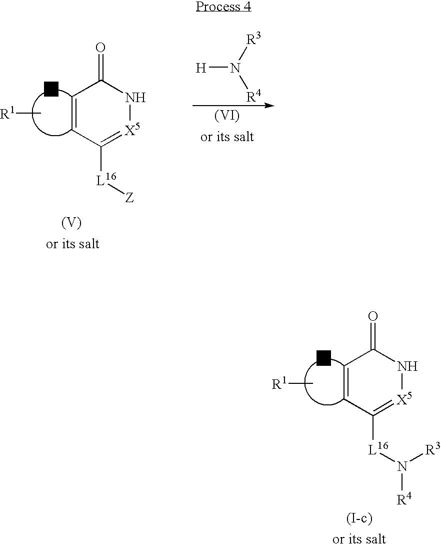
[wherein, R¹ and A are each as defined above, and X⁵ is CH or N, L¹⁶ has a same meaning of L¹² or L¹⁴, Z is halogen, and 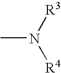
is substituted cyclic amino groups or optionally substituted amino group.]

This reaction can be carried out in the same manner as Process 3.

The compound of the present invention can be purified by any conventional purification methods employed for purifying organic compounds, such as recrystallization, column chromatography, thin-layer chromatography, high-performance liquid chromatography and the like. The compounds can be identified by conventional methods such as NMR spectrography, mass spectrography, IR spectrography, elemental analysis, and measurement of melting point.

Some of the starting compounds (II), (III), (V) and (V) are novel and can be prepared by the well-known processes or its analogous processes, for example, the processes described in the WO2000/42025 and the processes shown in Preparations mentioned below.

Suitable salts of the compounds of the present invention are pharmaceutically acceptable conventional non-toxic salts and can be an organic acid addition salt (e.g. formate, acetate, trifluoroacetate, maleate, tartrate, oxalate, methanesulfonate, benzenesulfonate, toluenesulfonate, etc.), an inorganic acid addition salt (e.g. hydrochloride, hydrobromide, sulfate, phosphate, etc.), a salt with an amino acid (e.g. aspartic acid salt, glutamic acid salt, etc.), or the like.

The “prodrug” means the derivatives of compounds of the present invention having a chemically or metabolically degradable group, which becomes pharmaceutically active after biotransformation.

The compounds of formula (I) may contain one or more asymmetric centers and thus they can exist as enantiomers or diastereoisomers. Furthermore certain compounds of formula (I) which contain alkenyl groups may exist as cis- or trans-isomers. In each instance, the invention includes both mixtures and separate individual isomers.

The compounds of the formula (I) may also exist in tautomeric forms and the invention includes both mixtures and separate individual tautomers.

The compound of the formula (I) and its salt can be in a form of a solvate, which is included within the scope of the present invention. The solvate preferably include a hydrate and an ethanolate.

Also included in the scope of invention are radiolabelled derivatives of compounds of formula (I) which are suitable for biological studies.

In the above and subsequent description of the present specification, suitable examples and illustrations of the various definitions, which the present invention includes within the scope thereof, are explained in detail as follows.

The term “lower” means a group having 1 to 6 carbon atom(s), unless otherwise provided.

Suitable “lower alkyl” includes a straight or branched alkyl having 1 to 6, in particular 1 to 2, carbon atoms. Preferable examples which may be mentioned are methyl, ethyl, propyl, isopropyl, butyl, isobutyl, tert-butyl, pentyl and hexyl.

Suitable “lower alkoxy” includes straight or branched alkoxy having 1 to 6, in particular 1 to 2, carbon atoms. Preferable examples which may be mentioned are methoxy, ethoxy, n-propoxy, isopropoxy, n-butoxy, iso-butoxy, sec-butoxy and tert-butoxy, preferably methoxy. Suitable “lower alkylamino” include mono (lower) alkylarnino and di (lower) alkylamino. Preferable examples which may be mentioned are methylamino, dimethylamino, ethylamino, dimethylamino, n-propylamino, isopropylamino, n-butylamino, iso-butylamino, sec-butylamino and tert-butylamino, preferably dimethylamino and diethylamino.

Suitable “lower alkylene” includes a straight or branched alkylene having 1 to 6, in particular 3, carbon atoms. Preferable examples which may be mentioned are methylene, ethylene, trimethylene, propylene, methyltrimethylene (1- or 2-methyltrimethylene) and hexamethylene, preferably trimethylene.

Suitable “lower alkenylene” includes a straight or branched alkenylene having 1 to 6, in particular 3, carbon atoms. Preferable examples which may be mentioned are vinylene, propenylene, dimethylpropenylene (e.g., 3,3-dimethylpropenylene, etc.) and hexenylene preferably propenylene.

The term “halogen” means fluoro, chloro, bromo or iodo.

Suitable “halo(lower)alkyl” contains 1 to 4, in particular 1 or 2, carbon atoms, and preferably 1 to 9, in particular 1 to 5, identical or different halogen atoms, preferably fluorine, chlorine and bromine, in particular fluorine and chlorine. Examples which may be mentioned are trifluoromethyl, trichloromethyl, chlorodifluoromethyl, dichlorofluoromethyl, chloromethyl, bromomethyl, 1-fluoroethyl, 2-fluoroethyl, 2,2-difluoroethyl, 2,2,2-trifluoroethyl, 2,2,2-trichloroethyl and pentafluoroethyl, preferably trifluoromethyl.

Suitable “halo(lower)alkoxy” contains 1 to 4, in particular 1 or 2, carbon atoms, and preferably 1 to 9, in particular 1 to 5, identical or different halogen atoms, preferably fluorine, chlorine and bromine, in particular fluorine and chlorine. Examples which may be mentioned are trifluoromethoxy, trichloromethoxy, chlorodifluoromethoxy, dichlorofluoromethoxy, chloromethoxy, bromomethoxy, 1 -fluoroethoxy, 2-fluoroethoxy, 2,2-difluoroethoxy, 2,2,2-trifluoroethoxy, 2,2,2-trichloroethoxy and pentafluoroethoxy, preferably trifluoromethoxy.

The term carbocyclic group intended to mean cyclo(oower)alkyl or cyclo(lower)alkenyl.

Suitable “cyclo(lower)alkyl” and cyclo(lower)alkyl moiety in the term “cyclo(lower)alkylene” includes a saturated carbocycle having 3 to 7, in particular 5 to 6, carbon atoms. Preferable examples which may be mentioned are cyclopropyl, cyclobutyl, cyclopentyl, cyclohexyl and cycloheptyl, preferably cyclopropyl and cyclohexyl.

Preferable example which may be mentioned as “cyclo(lower)alkylene” are cyclohexylene (e.g., 1,3-cyclohexylene, 1,4-cyclohexylene, etc.). Suitable “cyclooower)alkenyl” and cyclo(lower)alkenyl moiety in the term “cyclo(lower)alkenylene” includes a partially saturated carbocycle having 3 to 7, in particular 5 to 6, carbon atoms. Preferable examples which may be mentioned are cyclopropenyl, cyclobutenyl, cyclopentenyl, cyclohexenyl and cycloheptenyl, preferably cyclopentenyl and cyclohexenyl.

Preferable example which may be mentioned as “cyclo(lower)alkylene” are cyclopentenylene (e.g., 1,3-cyclocyclopent-1-enylene, etc.), cyclohexenylene (e.g., 1,3-cyclohex-1-enylene, etc.).

Suitable “heteroaryl” and heteroaryl moiety in the terms “heteroaryl(lower)alkyl” and “heteroaromatic acyl” is intended to mean 5- to 7-membered rings having preferably 1 to 3, in particular 1 or 2, identical or different heteroatoms. Heteroatoms in the heteroaryl are oxygen, sulfur or nitrogen. Examples which may be mentioned are furyl, thienyl, pyrazolyl, imidazolyl, triazolyl (e.g., 1,2,3- and 1,2,4-triazolyl, etc.), isoxazolyl, thiazolyl, isothiazolyl, oxadiazolyl (e.g., 1,3,4-, and 1,2,5-oxadiazolyl, etc.), azepinyl, pyrrolyl, pyridyl, piperazinyl, pyridazinyl, pyrimidinyl, pyrazinyl, triazinyl (e.g., 1,3,5-, 1,2,4- and 1,2,3-triazinyl, etc.), oxazinyl (e.g., 1,2,4- and 1,2,6-oxazinyl, etc.), oxepinyl, thiepinyl, diazepinyl (e.g., 1,2,4-diazepinyl, etc.), preferably thienyl, pyrazolyl, imidazolyl, thiazolyl, pyridyl, pyrazinyl.

Suitable “cyclic amino group” are heteroaromatic or aliphatic ring systems having one or more nitrogen atoms as the heteroatom, in which the heterocyclic rings can be saturated or unsaturated, can be one ring system or several fused ring systems, and optionally contain further heteroatoms, such as nitrogen, oxygen and sulfur and the like. Cyclic amino groups can furthermore also denote a spiro ring or a bridged ring system. The number of atoms which form cyclic amino groups is not limited, for example in the case of a single-ring system, they comprise 3 to 8 atoms, and in the case of a three-ring system, they comprise 7 to 11 atoms.

Preferable examples of “cyclic amino group” are described as follows:
(1) examples which may be mentioned of cyclic amino group with saturated monocyclic groups with one or more nitrogen atom(s) as the heteroatom are azetidinyl (3-azetidinyl), pyrrolidinyl (e.g., 1- and 3-pyrrolidinyl, etc.), piperidyl (e.g., piperidine, 4-piperidyl, etc.), homopiperidino (e.g., hexahydro-1H-azepin-1-yl, etc.), homopiperazinyl (e.g., hexahydro-1H-1, 4-diazepin-1-yl, etc.), imidazolidinyl (e.g., 1-imidazolidinyl, etc.), piperazinyl (e.g., 1-piperazinyl, etc.), perhydropyrimidinyl (e.g., perhydropyrimidin-1-yl, etc.) or diazacycloheptanyl (e.g., 1,4-diazacycloheptan-1-yl, etc.);
(2) examples which may be mentioned of cyclic amino group with unsaturated monocyclic groups with one or more nitrogen atom(s) as the heteroatom are pyrrolinyl (e.g., 2-pyrrolin-1-yl, etc.), pyrrolyl (e.g., 1-pyrrolyl, etc), tetrahydropyridyl (e.g., 3,6-dihydro-((2H)-pyridyl, etc.), pyridyl (e.g., 2-pyridyl, etc.), tetrahydroazepinyl (e.g., 2,3,6,7-tetrahydro-1H-azepin-1-yl, 2,3,4,7-tetrahydro-1H-azepin-1-yl, etc.), imidazolyl (1-imidazolyl), pyrazolyl, triazolyl, tetrazolyl, tetrazolyl, pyrimidinyl, pyrazinyl, pyridazinyl, dihydro-pyridazinyl (e.g., 1,2-dihydro-pyridazin-1-yl, etc.) or dihydro-pyrimidinyl (e.g., 1,2-dihydro-pyrimidin-1-yl, etc.);
(3) examples which may be mentioned of cyclic amino groups with saturated or unsaturated monocyclic groups with one to three nitrogen atoms and one to two sulfur atoms as heteroatoms are thiazolidinyl (e.g., 3-thiazolidinyl, etc.), isothiazolinyl (e.g., 2-isothiazolinyl, etc.) or thiomorpholino;
(4) examples which may be mentioned of cyclic amino groups with saturated or unsaturated monocyclic groups with one to three nitrogen atoms and one to two oxygen atoms as heteroatoms are oxazolyl, isoxazolyl, oxadiazolyl (e.g., 1,2,4-oxadiazolyl, or 1,3,4-oxadiazolyl) or morpholinyl;
(5) examples which may be mentioned of cyclic amino groups with saturated or unsaturated fused cyclic groups are indolyl (e.g., 1-indolyl, etc.), dihydrobenzimidazolyl (e.g., 1,2-dihydrobenzimidazol-1-yl, etc.), perhydropyrrolo[1,2-a]pyrazinyl (e.g., perhydropyrrolo[1,2-a]pyrazin-2-yl, etc.), tetrahydrobenzo[f]isoquinolinyl (e.g., 1,4,5,6-tetrahydrobenzo[f]isoquinolin-3(2H)-yl, etc.), hexahydrobenz[f]isoquinolinyl (e.g., cis- and trans-1,4,4a,5,6,10b-hexahydrobenz[f]isoquinolin-3(2H)-yl, etc.), tetrahydropyrido[3,4-b]indolyl (e.g., 1,3,4,9-tetaahydro-2H-pyrido[3,4-b]indol-2-yl, etc.) tetrahydrobenzazepinyl (e.g., 1,2,4,5-tetrahydro-3H-3-benzazepin-3-yl, etc.), or dihydroisoquinolinyl (e.g., 3,4-dihydro-2(1H)-isoquinolinyl, etc.);
(6) examples which may be mentioned of cyclic amino groups with spirocyclic groups are azaspiro[4,5]decanyl (e.g., 2-azaspiro[4,5]decan-2-yl, etc.), spiro[1H-indene-1,4′-piperidyl] (e.g., spiro[1H-indene-1,4′-piperidin-1′-yl], etc.), or dihydrospiro[1H-indene-1,4′-piperidyl] (e.g., 2,3-dihydrospiro[1H-indene-1,4′-piperidin-1′-yl], etc.);
(7) examples which may be mentioned of cyclic amino groups bridged heterocyclic groups are azabicyclo[2,2,1]heptanyl (e.g., 2-azabicyclo[2,21 ]heptan-7-yl, etc.), or diazabicyclo[2.2.1]heptyl (e.g., 2,5-diazabicyclo[2.2.1]hept-2-yl, etc.).

Among the above, preferable “cyclic amino group” included in R¹ is above-mentioned (1) or (2), in which the most preferable one is piperidyl, tetrahydropyridyl or piperazinyl.

Preferable examples which may be mentioned of “diradical of saturated or unsaturated monocyclic group with one or more nitrogen atom(s), which is obtained after removal of one hydrogen atom from said monocyclic group” are azetidinylene (e.g., 1,2- or 1,3-azetidinylene), pyrrolidinylene (e.g., 1,2- or 1,3-pyrrolidinylene), or piperidinylene (e.g., 1,3- or 1,4-piperidinylene).

It has been known that, during major cellular stresses, the activation of PARP can rapidly lead to cell damage or death through depletion of energy stores and PARP activation play a key role in both NMDA- and NO-induced neurotoxicity (Zhang et. al., Science, 263: 687-89 (1994)). Therefore, the compound possessing PARP inhibiting activity, such as the compound (I) of this invention, or pharmaceutically acceptable salts are useful in treating and preventing various diseases ascribed by NMDA- and NO-induced toxicity. Such diseases include, for example, tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Parkinson's disease; epilepsy; Amyotrophic lateral Scleosis (ALS); Huntington's disease; schizophrenia; chronic pain; ischenia and neuronal loss following hypoxia; hypoglycemia; ischemia; trauma; or nervous insult.

It has been demonstrated that PARP inhibitor are useful in deducing infarct size (Thiemermanm et al, Proc. Natl. Acad. Sci. USA, 94: 679-83 (1997)). Therefore, the compound possessing PARP inhibiting activity, such as the compound (I) of this invention, or pharmaceutically acceptable salts are useful in treatment and prevention of previously ischemic heart or skeleton muscle tissue.

It is also known that PARP is thought to play a role in enhancing DNA repair. So, the compound possessing PARP inhibiting activity, such as the compound (I) of this invention, or pharmaceutically acceptable salts are effective in treating and preventing radiosensitizing hypoxic tumor cells; tumor cells from recovering from potentially lethal damage of DNA after radiation therapy.

Further, the compound possessing PARP inhibiting activity, such as the compound (I) of this invention, or pharmaceutically acceptable salts are useful in extending the life-span and proliferative capacity of cells and altering gene expression of senescent cells. They are useful for treating and preventing skin aging; Alzheimer's diseases; arteriosclerosis; osteoarthritis; osteoporosis; muscular dystrophy; degenerative diseases of skeletal muscle involving replicative senescence; age-related macular degeneration; immune senescence; AIDS; and other immune senescence diseases.

Still further, the compound possessing PARP inhibiting activity, such as the compound (I) of this invention, or pharmaceutically acceptable salts are effective in treating and preventing inflammatory bowel disorders (e.g., colitis); arthritis; diabetes; endotoxic shock; septic shock; or tumor. Also, they are useful in reducing proliferation of tumor cells and making synergistic effect when tumor cells are co-treated with an alkylating drug.

The compound possessing PARP inhibiting activity, such as the compound (I) of this invention, or pharmaceutically acceptable salts are effective in treating and preventing pituitary apoplexy; conjunctivitis; retinoblastoma; retinopathy; acute retinal necrosis syndrome; Sjogren's syndrome.

The compound (I), its prodrug, or their salt can be administered alone or in the form of a mixture, preferably, with a pharmaceutical vehicle or carrier.

The active ingredient of this invention can be used in the form of a pharmaceutical preparation, for example, in solid, semisolid or liquid form, which contains a compound (I), as an active ingredient, in admixture with an organic or inorganic carrier or excipient suitable for external (topical), enteral, intravenous, intramuscular, parenteral or intramucous applications. The active ingredient can be formulated, for example, with the conventional non-toxic, pharmaceutically acceptable carriers for ointment, cream, plaster, tablets, pellets, capsules, suppositories, solution (saline, for example), emulsion, suspension (olive oil, for example), aerosols, pills, powders, syrups, injections, troches, cataplasms, aromatic waters, lotions, buccal tablets, sublingual tablets, nasal drops and any other form suitable for use. The carriers which can be used are water, wax, glucose, lactose, gum acacia, gelatin, mannitol, starch paster, magnesium trisilicate, talc, corn starch, keratin, paraffin, colloidal silica, potato starch, urea and other carriers suitable for use in manufacturing preparations, in solid, semisolid, or liquid form, and in addition auxiliary, stabilizing, thickening and coloring agents and perfumes may be used. The active compound is included in a pharmaceutical composition in an effective amount sufficient to produce the desired effect upon the process or condition of the diseases.

The active ingredient can be formulated into, for example, preparations for oral application, preparations for injection, preparations for external application, preparations for inhalation, preparations for application to mucous membranes.

Mammals which may be treated by the present invention include livestock mammals such as cows, horses, etc., domestic animals such as dogs, cats, rats, etc. and humans, preferably humans.

While the dosage of therapeutically effective amount of the compound (I) will vary depending upon the age and condition of each individual patient, an average single dose to a human patient of about 0.01 mg, 0.1 mg, 1 mg, 10 mg, 50 mg, 100 mg, 250 mg, 500 mg, and 1000 mg of the compound (I) may be effective for treating the above-mentioned diseases. In general, amounts between 0.01 mg/body and about 1,000 mg/body may be administered per day.

In order to illustrate the usefulness of the object compound (I), the pharmacological test data of the compound (I) are shown in the following.

A. Test Compound

(1) 2-[3-(4-Phenyl-3,6-dihydro-1(2H)-pyridyl)propyl]-5,6,7,8-tetrahydro-4(3H)-quinazolinone
(Compound A: The compound of Example 1)

(2) 2-[3-(4-Phenyl-3,6-dihydro-1(2H)-pyridyl)propyl]-3,5,7,8-tetrahydro4H-thiopyrano[4,3-d]pyrimidin-4-one
(Compound B: The compound of Example 3-(10))

(3) 4-[4-(4-Phenyl-3,6dihydro-1(2H)-pyridyl)butyl]-1(2H)-phthalazinone
(Compound C: The compound of Example 7)

(4) 4-[4-(9-Methyl-1,3,4,9-tetrahydro-2H-pyrido[3,4-blindol-2-yl)butyl)-1(2H)-phthalazinone
(Compound D: The compound of Example 9-(7))
   B. PARP Inhibitory Activity (In vitro assay)
   (1) Assay Conditions:

The recombinant human PARP (5.3 mg protein/ml) were incubated with a test compound in a 100 μl reaction buffer containing the indicated concentration of 1 mCi/ml ³²P-NAD, 50 mM Tris-HCl, 25 mM MgCl₂, 1 mM DTT (dithiothreitol), 0.05 mM NAD (nicotinamido adenine dinucleotide), lmg/mi activated DNA, pH8.0. Incubation was for 15 minutes at a room temperature and the reaction was stopped by the addition of 200 μl of ice-cold 20% trichloroacetic acid followed by rapid filtration through GF/B filters. The filters were treated with scintillation fluid and acid-insoluble counts were measured for quantification of unit activity.

PARP Inhibitory Activity (%)=

[1-(enzyme activity with test compound)/(enzyme activity with vehicle)]×100

(2) Result

PARP Inhibitory Activity (IC₅₀) in Test Compound.

| Test Compound | IC50(μM) |
|---|---|
| Compound A | <0.5 |
| Compound B | <0.5 |
| Compound C | <0.5 |
| Compound D | <0.5 |

This invention relates to novel Quinazolinone compounds had a potent PARP inhibitory activity. PARP inhibitors including this invention relates to novel quinazolinone compounds were effective in preventing reduction of striatal DA and its metabolite induced by MPTP treatment in mice. Therefore, it suggests that these compounds may have protective benefit in the treatment of neurodegenerative disease such as Parkinson's disease.

Abbreviations used herein have the following meanings:

| ABBREVIATION | DEFINITION |
|---|---|
| Me | methyl |
| Et | ethyl |
| TBu | tert-buthyl |
| Bzl | benzyl |
| Ph | phenyl |
| Ac | acetyl |
| Bz | benzoyl |

Any patents, patent applications, and publications cited herein are incorporated by reference.

### BEST MODE FOR CARRYING OUT THE INVENTION

The following Preparation and Examples are given for the purpose of illustrating the present invention in detail, but are not to be construed to limit the scope of the present invention.

Preparation 1

To a solution of 3,4-difluorobromobenzene (5.81 g) in tetralydrofuran (50 ml) was added dropwise n-butyl lithium (19.3 ml) at −78° C. under nitrogen. The mixture was stirred at the temperature for 0.5 hour. To the mixture was added dropwise a solution of t-Butyl 4-oxo-1-piperidinecarboxylate (5 g) in tetrahydrofuran (20 ml) at −78° C., and the mixture was stirred for 1 hour, then warmed to 0° C. and stirred for further 1 hour. The reaction was quenched with water and extracted with ethyl acetate twice. The combined extracts were dried over magnesium sulfate and concentrated. This crude t-Butyl 4-(3,4-difluorophenyl)-4-hydroxy-1-piperidinecarboxylate was used for the next step without further purification.

Preparation 2

To a solution of t-butyl 4-(3,4-difluorophenyl)-4-hydroxy-1 -piperidinecarboxylate (8.96 g; net: 7.79 g) in dichloromethane (98 ml) were added in sequence methanesulfonylchloride (5.77 ml), triethylamine (34.7 ml) and 4-dimethylaminopyridine (152 mg). After stirring at room temperature for 2 hours, the mixture was diluted with water and extracted with dichloromethane twice. The combined extracts were dried over magnesium sulfate and concentrated. A solution of the residue and triethylamine (34.7 ml) in dichloromethane (98 ml) was stirred at room temperature for 2 days. The mixture was diluted with water and the organic layer was separated. The organic extract was dried over magnesium sulfate and concentrated. The residue was chromatographed on silica gel using 10% ethyl acetate in hexane as an eluent to give t-Butyl 4-(3,4-difluorophenyl)-3,6-dihydro-1(2H)-pyridinecarboxylate (4.37 g) as an oil.

¹H NMR (CDCl₃, δ): 1.50 (9H, s), 2.40-2.60 (2H, m), 3.63 (2H, t, J=5.7 Hz), 3.90-4.20 (2H, m), 5.97 (1H, s), 6.80-7.40 (4H, m).

Mass (ESI): 318.2 (M+Na)³⁰

Preparation 3

To a solution of t-butyl 4-(3,4-difluorophenyl)-3,6-dihydro-1(2H)-pyridinecarboxylate (4.3 g) in ethyl acetate (20 ml) was added dropwise 4N hydrogen chloride in ethyl acetate (18.25 ml), and the mixture was stirred at room temperature overnight. After evaporation of the mixture, the residue was triturated with ethyl acetate and diisopropylether, and the resulting powder was collected, washed with diisopropylether and dried in vacuo to give 4-(3,4-Difluorophenyl)-1,2,3,6-tetrahydropyridine hydrochloride (3.25 g).

¹H NMR (DMSO-d₆, δ): 2.20-4.20 (6H, m), 6.09 (1H, s), 7.00-7.80 (3H, m), 9.07 (2H, brs)

Mass (ESI): 196.2 (M+H)⁺

Preparation 4

To a suspension of L-alanine methyl ester hydrochloride (12.9 g) and triethylamine (38.6 ml) in dichloromethane (130 ml) was added dropwise chloroacetylchloride (8.83 ml) at 0° C. After stiring at 0° C. for 30 minutes, the mixture was concentrated and diluted with ethyl acetate (100 ml) and 1N aqueous hydrochloric acid (100 ml). The organic layer was separated, washed with water twice, dried over magnesium sulfate and concentrated. A solution of the residue in 40% ethyl acetate in hexane (200 ml) was treated with silica gel (85 g), and silica gel was removed by filtration and washed with 40% ethyl acetate in hexane (200 ml) twice, and the combined filtrate was concentrated to give methyl (2S)-2-[(chloroacetyl)arnino]propanoate as a brown oil.

¹H NMR (DMSO-d₆, δ): 1.30 (3H, d, J=7.3 Hz), 3.64 (3H1, s), 4.09 (2H, s), 4.20-4.35 (1H, m), 8.64 (1H, d, J=6.8 Hz)

Mass (ESI): 202.2 (M+Na)³⁰

Preparation 5

A solution of methyl (2S)-2-[(chloroacetyl)amino]propanoate (5 g), 4chloroaniline (3.55 g) and triethylamine (11.6 ml) in toluene (50 ml) was stirred at 100° C. overnight. The mixture was diluted with water (100 ml) and extracted with ethyl acetate twice. The combined extracts were washed with water and brine, dried over magnesium sulfate and concentrated. The residue was chromatographed on silica gel using 50% ethyl acetate in hexane as an eluent to give methyl (2S)-2-({[(4-chlorophenyl)amino]acetyl}amino)propanoate (3.07 g) as an oil.

¹H NMR (DMSO-d₆, δ): 1.27 (3H, d, J=7.3 Hz), 3.31 (3H, s), 3.66 (2H, d, J=6.0 Hz), 4.20-4.50 (1H, m), 6.12 (1H, t, J=6.0 Hz), 6.54 (2H, d, J=8.8 Hz), 7.1 (2H, d, J=8.8 Hz), 8.32 (1H, d, J=7.2 Hz).

Mass (ESI): 293.2 (M+Na)³⁰

Preparation 6

A slurry of methyl (2S)-2-({[(4-chlorophenyl)amino]acetyl}amino)propanoate (3.02 g) and potassium t-butoxide (2.5 g) in toluene was stirred at 80° C. overnight. After cooling to room temperature, the reaction was quenched with 1N aqueous hydrochloric acid and extracted with ethyl acetate twice. The combined extracts were dried over magnesium sulfate and concentrated. The residue was chromatographed on silica gel using 80% ethyl acetate in hexane as an eluent to give (3S)-1-(4-Chlorophenyl)-3-methyl-2,5-piperazinedione (1.5 g).

¹H NMR (DMSO-d_{4, δ):} 1.37 (3H, d, J=7.0 Hz), 4.11 (1H, q, J=7.0 Hz), 4.22 (1H, d, J=16.6 Hz), 4.32 (1H, d, J=16.6 Hz), 7.30-7.60 (4H, m), 8.41 (1H, brs)

Mass (ESI): 261.1 (M+Na)³⁰

Preparation 7

The following compound was prepared in a similar manner to that of Preparation 4.

(1) Ethyl 2-((chloroacetyl)amino)-2-methylpropanoate

¹H NMR (DMSO-d₆, δ): 1.14 (3H, t, J=7.1 Hz), 1.36 (6H, s), 3.80-4.20 (4H, m), 8.52 (1H, brs)

Mass (ESI): 230.2 (M+Na)³⁰

Preparation 8

The following compound was prepared in a similar manner to that of Preparation 5.

(1) Ethyl 2-({[(4-chlorophenyl)amino]acetyl}amino)-2-methylpropanoate

¹H NMR (DMSO-d₆, δ): 1.10 (3H, t, J=7.1 Hz), 1.35 (6H, s), 3.61 (2H, d, J=6.0 Hz), 4.00 (2H, q, J=7.1 Hz), 6.54 (2H, d, J=8.8 Hz), 7.09 (2H, d, J=8.8 Hz), 8.17 (1H, brs)

Mass (ESI): 321.2 (M+Na)⁺

Preparation 9

The following compound was prepared in a similar manner to that of Preparation 6.

(1) 1-(4-Chlorophenyl)-3,3-dimethyl-2,5-piperazinedione

¹H NMR (DMSO-d₆, δ): 1.42 (6H, s), 4.32 (2H, s), 7.20-7.70 (4H, m), 8.50 (1H, brs)

Mass (ESI): 275.1 (M+Na)³⁰

Preparation 10

To a suspension of lithium aluminum hydride (225 mg) in tetrahydrofuran (7.5 ml) was added in portions 1-(4-chlorophenyl)-3,3-dimethyl-2,5-piperazinedione (0.5 g), and the mixture was stirred at 50° C. for 3 hours. After cooling to room temperature, the reaction was quenched with 1N aqueous sodium hydroxide (0.5 ml). The resulting precipitates were removed by filtration and washed with ethyl acetate, and then the combined filtrate was washed with brine, dried over magnesium sulfate and concentrated. A solution of the residue in ethyl acetate was treated with 4N hydrogen chloride in ethyl acetate (1 ml), and the mixture was concentrated. The residual oil was triturated with a small amount of acetone, and then the resulting powder was collected, washed with acetone and dried in vacuo to give 1-(4-Chlorophenyl)-3,3-dimethylpiperazine hydrochloride (0.22 g).

¹H NMR (DMSO-d₆, δ): 1.37 (6H, s), 3.00-3.40 (6H, m), 7.02 (2H, d, J=9.0 Hz), 7.28 (2H, d, J=9.0 Hz), 9.08 (2H, brs)

Mass (ESI): 225.3 (M+H)⁺

Preparation 11

The following compound was prepared in a similar manner to that of Preparation 10.

(1) (3S)-1-(4Chlorophenyl)3-methylpiperazine hydrochloride

¹H NMR (DMSO-d₆, δ): 1.29 (3H, d, J=6.5 Hz), 1.80-4.30 (7H, m), 6.90-7.40 (4H, m)

Mass (ESI): 211.2 (M+H)⁺

Preparation 12

A mixture of 4-bromochlorobenzene (2 g), 2-amino-2-methyl-1-(triphenylmethyl)aminopropane (4.83 g), tris(dibenzylideneacetone)dipalladium (287 mg), 2,2′-bis(diphenylphosphino)-1,1′-binaphthyl (390 mg), sodium t-butoxide (1.4 g) in toluene (24 ml) was stirred at 120° C. under nitrogen for 2 hours. After cooling to room temperature, the mixture was diluted with diisopropylether and filtered, and the filtrate was concentrated. The residue was chromatographed on silica gel using 10% ethyl acetate in hexane as an eluent to give 2-(4-chlorophenyl)amino-2-methyl-1-(triphenylmethyl)amino-propane (2.83 g).

¹H NMR (CDCl₃, δ): 1.30 (6H, s), 1.92 (1H, t, J=6.8 Hz), 2.27 (1H, d, J=6.8 Hz), 3.59 (1H, brs), 6.26 (2H, d, J=8.8 Hz), 6.91 (2H, d, J=8.8 Hz), 7.10-7.70 (15H, m)

Mass (ESI): 463.3 (M+Na)³⁰

Preparation 13

To a solution of 2-(4-chlorophenyl)amnino-2-methyl-1-(triphenylnethyl)amino-propane (2.79 g) in dichloromethane (100 ml) were added in sequence triethylamine (3.88 ml) and methyl oxalyl chloride (1.16 ml). After stirring at room temperature for 4 hours, the mixture was washed with sodium hydrogen carbonate aqueous solution, dried over magnesium sulfate and concentrated. The residue was chromatographed on silica gel (ethyl acetate/hexane=1/9 to 1/1) to give methyl {(4-chlorophenyl)-[1,1-dimethyl-2-((triphenylmethyl)amnino)ethyl]amino}(oxo)acetate (3.3 g) as an oil.

¹H NMR (CDCl₃, δ): 1.28 (6H, s), 1.87 (1H, t, J=8.5 Hz), 2.63 (2H, d, J=8.5 Hz), 3.46 (3H, s), 7.10-7.70 (19H, m)

Mass (ESI): 549.3 (M+Na)³⁰

Preparation 14

To a solution of methyl {(4-chlorophenyl)-[1,1-dimethyl-2-((triphenylmethyl)amino)ethyl]amino}(oxo)acetate (3.3 g) in dichloromethane were added in sequence anisole (3.3 ml) and trifluoroacetic acid (6 ml) at 0° C. After stirring at this temperature for 2 hours, the mixture was diluted with water and extracted with dichloromethane twice. The combined extracts were dried over magnesium sulfate and concentrated. A suspension of the residue in 2-propanol (15 ml) was stirred at 80° C. in the presence of acetic acid (1 ml) for 2 hours. The mixture was cooled to 0° C., and the resulting precipitates were collected, washed with 2-propanol and dried in vacuo (40° C.) to give 1-(4-chlorophenyl)-6,6-dirnethyl-2,3-piperazinedione (1.17 g).

¹H NMR (CDCl₃, δ): 1.34 (6H, s), 3.55 (2H, d, J=3.3 Hz), 7.00-7.20 (3H, m) 7.43 (2H, d, J=8.6 Hz)

Mass (ESI): 275.2 (M+Na)³⁰

Preparation 15

To a suspension of 1-(4-chlorophenyl)-6,6-dimethyl-2,3-piperazinedione (0.69 g) in tetrahydrofuran (25 ml) was added dropwise 2M boran-methyl sulfide complex in tetrahydrofuran (6.8 ml) under nitrogen, and the mixture was stirred at room temperature overnight. The reaction was quenched with methanol and 12N aqueous hydrochloric acid (1.5 ml) was added. After stirring at 70° C. for 1 hour, the mixture was cooled to room temperature, basified with 1N aqueous sodium hydroxide and extracted with dichloromethane twice. The combined extracts were dried over magnesium sulfate and concentrated. The residue was dissolved in dichloromethane, treated with 4N hydrogen chloride in ethyl acetate (1 ml) and concentrated to give 1-(4-Chlorophenyl)-2,2-dimethylpiperazine hydrochloride (0.46 g) as an amorphous powder.

¹H NMR (DMSO-d₆, δ): 1.09 (6H, s), 2.90-3.40 (6H, m), 7.20 (2H, d, J=8.7 Hz), 7.38 (2H, d, J=8.7 Hz), 9.38 (2H, brs)

Mass (ESI): 225.3 (M+H)⁺

Preparation 16

A mixture of 4-bromochlorobenzene (1.5 g), cis-2,6-dimethylpiperazine (1.07 g), trnns-dichlorobis(tri-o-tolylphosphine)palladium (II) (185 mg), sodium t-butoxide (1.09 g) in toluene (20 ml) was stirred at 100° C. under nitrogen for 3 hours. After cooling to room temperature, the reaction was quenched with water and extracted with dichloromethane twice. The combined extracts were dried over magnesium sulfate and concentrated. The residue was chromatographed on silica gel using 5% methanol in dichloromethane as an eluent to give 4-(4-Chlorophenyl)-cis-2,6-dimethylpiperazine (1.46 g) as a solid.

Mass ESI): 225.3 (M+H)⁺

Preparation 17

A biphasic solution of (3R,5R)-1-benzyl-3,5-dimethylpiperazine (1.61 g; net: 1.50 g) and di-t-butyldicarbonate (1.61 g) in dichloromethane (20 ml) and 1N aqueous sodium hydroxide (20 ml) was stirred at room temperature for 30 minutes. The organic phase was separated and the aqueous layer was flither extracted with dichloromethane. The combined extracts were dried over magnesium sulfate and concentrated in vacuo. The residue was dissolved in 20% ethyl acetate in hexane and treated with silica gel (7.5 g). Silica gel was removed by filtration and washed with 20% ethyl acetate in hexane twice, and then the combined filtrate was evaporated to afford colerless oil. A solution of the residue in methanol was hydrogenated over 10% palladium-on-charcoal (450 mg) for 3 hours. The catalyst was removed by filtration and the filtrate was concentrated. The residue was chromatographed on silica gel (20% ethyl acetate in hexane to 10% methanol in dichloromethane), and then the fractions eluted with 10% methanol in dichloromethane were combined and concentrated to give t-butyl (2R,6R)-2,6-dimethyl-1-piperazinecarboxylate (1.32 g) as an oil.

¹H NMR (CDCl₃, δ): 1.30 (6H, d, J=6.6 Hz), 1.47 (9H, s), 2.71 (2H, dd, J=4.4, 12.6 Hz), 3.15 (2H, dd, J=4.0, 12.6 Hz), 3.70-4.00 (2H, m)

Mass (ESI): 237.3 (M+Na)³⁰

Preparation 18

A mixture of t-butyl (2R,6R)-2,6-dimethyl-1-piperazinecarboxylate (1.27 g), 4-bromochlorobenzene (3.4 g), tris(dibenzylideneacetone)dipalladium (0) (271 mg), 2,2′-bis(diphenylphosphino)-11′-binaphthyl (369 mg), sodium t-butoxide (2.28 g) in toluene (26 ml) was stirred at 80° C. under nitrogen overnight. The mixture was cooled, diluted with water and extracted with dichloromethane twice. The combined extracts were dried over magnesium sulfate and concentrated. The residue was dissolved in 20% ethyl acetate in hexane (50 ml) and treated with silica gel (20 g). Silica gel was removed by filtration and washed with 20% ethyl acetate in hexane (50 ml) twice, and then the combined filtrate was evaporated. To a solution of the residue in dichloromethane (30 ml) was added dropwise trifluoroacetic acid at 0° C. After stirring for 1 hour, the mixture was concentrated, basified with 1N aqueous sodium hydroxide and extracted with dichloromethane twice. The combined extracts were dried over magnesium sulfate and concentrated. The residue was chromatographed on silica gel (30 g) (50% ethyl acetate in hexane to 10% methanol in dichloromethane), and the fractions eluted with 10% methanol in dichloromethane were combined and concentrated. A solution of the residue in ethyl acetate was treated with 4N hydrogen chloride in ethyl acetate (2 ml), and the resulting powder was collected, washed with ethyl acetate and dried in vacuo to give (3R,5R)-1-(4-Chlorophenyl)-3,5ethylpiperazine hydrochloride (1.49 g).

¹H NMR (DMSO-d₆, δ): 1.34 (6H, d, J=6.6 Hz), 3.12 (2H, dd, J=6.4, 13.0 Hz), 3.43 (2H, dd, J=3.3, 13.0 Hz), 6.99 (2H, d, J=9.0 Hz), 7.27 (2H, d, J=9.0 Hz), 9.48 (2H, brs) Mass(ESI): 225.3 (M+H)⁺

Preparation 19

The following compound was prepared in a similar manner to that of Preparation 17.

(1) t-Butyl (2S,6S)-2,6-dimethyl-1-piperazinecarboxylate

¹H NMR (CDCl₃, δ): 1.30 (6H, d, J=6.6 Hz), 1.47 (9H, s), 2.71 (2H, dd, J=4.4, 12.6 Hz), 3.15 (2H, dd, J=4.0, 12.6 Hz), 3.70-4.00 (2H, m)

Mass (ESI): 237.3 (M+Na)³⁰

Preparation 20

The following compound was prepared in a similar manner to that of Preparation 18.

(1) (3S,5S)-1-(4-Chlorophenyl)-3,5-dimethylpiperazine hydrochloride

¹H NMR (DMSO-d₆, δ): 1.34 (6H, d, J=6.6 Hz), 3.12 (2H, dd, J=6.4, 13.0 Hz), 3.43 (2H, dd, J=3.3, 13.0 Hz), 6.99 (2H, d, J=9.0 Hz), 7.27 (2H, d, J=9.0 Hz), 9.48 (2H, brs)

Mass (ES1): 225.3 (M+H)⁺

Preparation 21

A mixture of 4-phenyl-1,2,3,6-tetrahydropyridine hydrochloride (6 g), 4-bromobutyronitrile (3.35 ml) and diisopropylethylamine (16 ml) in N,N-dimethylformamide (30 ml) was stirred at 80° C. for 3 hours. The mixture was diluted with water, extracted with ethyl acetate twice. The combined extracts were washed with water three times, dried over magnesium sulfate and concentrated. The residue was dissolved in ethyl acetate and treated with silica gel (30 g). Silica gel was removed by filtration and washed with ethyl acetate. The combined fitrate was concentrated to give 4-(4-phenyl-3,6dihydro-1(2H)-pyridyl)butanenitrile as an oil.

¹H NMR (CDCl₃, δ): 1.75-2.10 (2H, m), 2.30-2.90 (8H, m), 3.05-3.25 (2H, m), 6.06 (1H, s), 7.10-7.80 (5H, m)

Mass (APCI): 227.40 (M+H)⁺

Preparation 22

To a suspension of ammonium chloride (2.95 g) in toluene (20 ml) was added dropwise 2N trimethylaluminium in toluene (27.5 ml) at 0° C. under nitrogen, and the mixture was stirred at room temperature for 2 hours. To this aluminum amide reagent was added dropwise 4-(4-phenyl-3,6-dihydro-1(2H)-pyridyl)butanenitrile (2.5 g) in toluene (10 ml) at room temperature, and this solution was stirred at 80° C. overnight. The reaction mixture was carefully poured into a suspension of silica gel (60 g) in chloroform (180 ml). Silica gel was removed by filtration and washed with methanol (200 ml), and then the combined filtrate was concentrated. The residue was chromatographed on aluminum (68 g) using 20% methanol in dichloromethane as an eluent to give 4-(4-phenyl-3,6-dihydro-1(2H)-pyridyl)butanimidamide (2.04 g) as an oil.

¹H NMR (DMSO-d₆,δ):1.70-2.00 (2H, m), 2.10-2.90 (8H, m), 3.09 (2H, d, J=2.8 Hz), 6.16 (1H, s), 7.10-7.70 (5H, m), 8.69 (3H, brs)

Mass (APCI): 244.33 (M+H)⁺

Preparation 23

The following compounds were prepared in a similar manner to that of Preparation 21.

(1) 4-[4-(3,4-Difluorophenyl)-3,6-dihydro-1(2H)-pyridyl]butanenitrile

¹H NMR (DMSO-d₆, δ): 1.60-2.00 (2H, m), 2.20-2.80 (8H, m), 3.07 (2H, d, J=2.6 Hz), 6.04 (1H, s), 7.00-7.80 (3H, m)

Mass (EST): 263.3 (M+H)⁺

(2) 4-[4-(4-Chlorophenyl)-2,2-dimethyl-1-piperazinyl]butanenitrile

¹H NMR (DMSO-d₆, δ): 1.08 (6H, s), 1.50-1.80 (2H, m), 2.20-2.70 (6H, m), 2.87 (2H, s), 3.00-3.20 (2H, m), 6.91 (2H, d, J=9.1 Hz), 7.20 (2H, d, J=9.1 Hz)

Mass (ESI): 292.3 (+H)⁺

(3) 4-[(2S)4-(4-Chlorophenyl)-2-methyl-1-piperazinyl]butanenitrile

¹H NMR (DMSO-d₆, δ): 1.05 (3H, d, J=5.6 Hz), 1.60-1.90 (2H, m), 2.00-3.60 (11H, m), 6.93 (2H, d, J=9.1 Hz), 7.21 (2H, d, J=9.1 Hz)

Mass (ESI): 278.2 (M+H)⁺

(4) 4-[4-(4-Chlorophenyl)-3,3dimethyl-1-piperazinyl]butanenitrile

¹H NMR (DMSO-d₆, δ): 0.98 (6H, s), 1.60-1.90 (2H, m), 2.20-3.20 (10H, m), 7.10 (2H, d, J=8.8 Hz), 7.29 (2H, d, J=8.8 Hz)

Mass (ESI): 292.4 (M+H)⁺

(5) 4-[(2R,6S)-4(4-Chlorophenyl)-2,6-dimethyl-1-piperazinyl]butanenitrile

¹H NMR (CDCl₃, δ): 1.16 (6H, s), 1.60-3.60 (12H, m), 6.82 (2H, d, J=9.0 Hz), 7.19 (2H, d, J=9.0 Hz)

Mass (ESI): 292.4 (M+H)⁺

(6) 4-[(2R,6R)-4-(4-Chlorophenyl)-2,6-dimethyl-1-piperazinyl]butanenitrile

¹H NMR (DMSO-d₆, δ): 1.01 (6H, d, J=6.1 Hz), 1.50-1.80 (2H, m), 2.20-3.30 (10H, m), 6.91 (2H, d, J=9.0 Hz), 7.20 (2H, d, J=9.0 Hz)

Mass (ESI): 292.2 (M+H)⁺

(7) 4-[(2S,6S)-4-(4-Chlorophenyl)-2,6-dimethyl-1-piperazinyl]butanenitrile

¹H NMR (DMSO-d₆, δ): 1.01 (6H, d, J=6.1 Hz), 1.50-1.80 (2H, m), 2.20-3.30 (10H, m), 6.91 (2H, d, J=9.0 Hz), 7.20 (2H, d, J=9.0 Hz)

Mass (ESI): 292.2 (M+H)⁺

(8) 4-[4-(4-Fluorophenyl)-3,6-dihydro-1(2H)-pyridyl]butanenitrile

¹H NMR (DMSO-d₆, δ): 1.60-2.00 (2H, m), 2.20-2.80 (8H, m), 3.06 (2H, d, J=3.0 Hz), 6.12 (1H, t, J=3.0 Hz), 7.00-7.70 (4H, m)

Mass (ESI): 245.4 (M+H)⁺

(9) 4-[4-(4-chlorophenyl)-3,6-dihydro-1(2H)-pyridyl]butanenitrile

¹H NMR (DMSO-d₆, δ): 1.60-1.90 (2H, m), 2.30-3.20 (10H, m), 6.19 (1H, t, J=3.5 Hz), 7.30-7.70 (4H, m)

Mass (APC1): 261.07 (M+H)⁺

(10) 4-[4-(4-Methylphenyl)-3,6dihydro-1(2H)-pyridyl]butanenitrie

¹H NMR (DMSO-d₆, δ): 1.60-1.90 (2H, m), 2.28 (3H, s), 2.30-2.80 (81H, m), 3.07 (2H, d, J=2.7 Hz), 6.09 (1H, s, J=2.7 Hz), 7.13 (2H, d, J=8.0 Hz), 7.31 (2H, d, J=8.0 Hz)

Mass (APCI): 241.33 (M+H)⁺

(11) 4-[4-(4-Trifluoromethylphenyl)-3,6-dihydro-1(2H)-pyridyl]butanenitrile

¹H NMR (DMSO-d₆, δ): 1.70-2.00 (2H, m), 2.30-3.20 (10H, m), 6.33 (1H, s), 7.50-7.70 (4H, m)

Mass (APCI): 295.00 (M+H)⁺

(12) 4-[4-(4-Methoxyphenyl)-3,6-dihydro-1(2H)-pyridyl]butanenitrile

¹H NMR (DMSO-d₆, δ): 1.70-2.00 (2H, m), 2.30-2.80 (8H, m), 3.74 (3H, s), 6.03 (1H, s), 6.89 (2H, d, J=8.8 Hz), 7.36 (2H, d, J=8.8 Hz)

Mass (APCI): 257.27 (M+H)⁺

(13) 4-[4-(4-Chlorophenyl)-1-piperazinyl]butanenitrile

¹H NMR (DMSO-d₆, δ):1.70-1.90 (2H, m), 2.30-2.80 (8H, m), 3.12 (4H, t, J=5.0 Hz), 6.94 (2H, d, J=9.1 Hz), 7.22 (2H, d, J=9.1 Hz)

Mass (APCI): 264.47 (M+H)⁺

(14) 4-[4-(4-Fluorophenyl)-1-piperazinyl]butanenitrile

¹H NMR (DMSO-d₆, δ): 1.60-2.00 (2H, m), 2.30-2.80 (8H, m), 3.07 (4H, t, J=5.0 Hz), 6.80-7.20 (4H, m)

Mass (ESI): 248.3 (M+H)⁺

(15) 4-[4-(4—Nitrophenyl)-1-piperazinyl]butanenitrile

¹H NMR (DMSO-d₆, δ): 1.70-1.90 (2H, m), 2.20-2.80 (8H, m), 3.45 (4H, t, J=5.0 Hz), 7.03 (2H, d, J=9.4 Hz), 8.05 (2H, d, J=9.4 Hz)

Mass (ESI): 275.3 (M+H)⁺

Preparation 24

The following compounds were prepared in a similar manner to that of Preparation 22.

(1) 4-[4-(3,4-Difluorophenyl)-3,6-dihydro-1(2H)-pyridyl]butanimidamide

¹H NMR (DMSO-d₆, δ): 1.70-2.00 (2H, m), 2.20-3.30 (10H, m), 6.05 (1H, s), 7.00-7.70 (3H, m)

Mass (ESI): 280.4 (M+H)⁺

(2) 4-[4-(4-Chlorophenyl)-2,2-diinethyl-1-piperazinyl]butanimidamide

¹H NMR (DMSO-d₆, δ): 1.03 (6H, s), 1.50-1.90 (2H, m), 2.20-3.30 (10H, m), 6.92 (2H, d, J=9.0 Hz), 7.21 (2H, d, J=9.0 Hz), 8.45 (3H, brs)

Mass (ESI): 309.3 (M+H)⁺

(3) 4-[(2S)-4-(4-Chlorophenyl)-2-methyl-1-piperazinyl]butanimidamide

¹H NMR (DMSO-d₆, δ): 1.04 (3H, d, J=5.5 Hz), 1.60-2.00 (2H, m), 2.00-3.70 (11H, m), 6.93 (2H, d, J=9.0 Hz), 7.22 (2H, d, J=9.0 Hz), 8.68 (3H, brs)

Mass (ESI): 295.4 (M+H)⁺

(4) 4-[4-(4-Chlorophenyl)3,3-dimethyl-1-piperazinyl]butanimidamide

¹H NMR (DMSO-d₆, δ): 0.99 (6H, s), 1.60-1.90 (2H, m), 2.10-3.20 (10H, m), 7.10 (2H, d, J=8.8 Hz), 7.30 (2H, d, J=8.8 Hz), 9.03 (3H, brs)

Mass (ESI): 309.3 (M+H)⁺

(5) 4-[(2R,6S)-4-(4Chlorophenyl)-2,6-dimethyl-1-piperazinyl]butanimidamide

¹H NMR (DMSO-d₆, δ): 1.06 (6H, d, J=6.2 Hz), 1.50-1.90 (2H, m), 2.10-3.90 (10H, m), 6.92 (2H, d, J=9.1 Hz), 7.21 (2H, d, J=9.1 Hz)

Mass (ESI): 309.3 (M+H)⁺

(6) 4-[(2R,6R)-4-(4-Chlorophenyl)-2,6-dimethyl-1-piperazinyl]butanimidamide

¹H NMR (DMSO-d₆, δ): 1.01 (6H, d, J=6.1 Hz), 1.50-1.90 (2H, m), 2.20-3.30 (10H, m), 6.92 (2H, d, J=9.0 Hz), 7.21 (2H, d, J=9.0 Hz), 8.79 (3H, brs)

Mass (ESI): 309.3 (M+H)⁺

(7) 4-[(2S,6S)-4-(4-Chlorophenyl)-2,6-dimethyl-1-piperazinyl]butanimidamide

¹H NMR (DMSO-d₆, δ): 1.01 (6H, d, J=6.1 Hz), 1.50-1.90 (2H, m), 2.20-3.30 (10H, m), 6.92 (2H, d, J=9.0 Hz), 7.21 (2H, d, J=9.0 Hz), 8.79 (3H, brs)

Mass (ESI): 309.3 (M+H)⁺

(8) 4-[4-(4-Fluorophenyl)-3,6-dihydro-1(2H)-pyridyl]butanimidamide

¹H NMR (DMSO-d₆, δ): 1.70-2.00 (2H, m), 2.30-2.80 (8H, m), 3.08 (2H, d, J=2.9 Hz), 6.13 (11H, s), 7.10-7.60 (4H, m)

Mass (ESI): 262.4 (M+H)⁺

(9) 4-[4-(4-Chlorophenyl)-3,6-dihydro-1(2H)-pyridyl]butanimidamide

¹H NMR (DMSO-d₆, δ):1.70-2.00 (2H, m), 2.20-2.80 (8H, m), 3.09 (2H, d, J=2.8 Hz), 6.21 (1H, s), 7.20-7.60 (4H, m)

Mass (APCI): 278.07 (M+H)⁺

(10) 4-[4-(4-Methylphenyl)-3,6-dihydro-1(2H)-pyridyl]butanimidamide

¹H NMR (DMSO-d₆, δ): 1.70-2.00 (2H, m), 2.28 (3H, s), 2.30-2.70 (8H, m), 3.08 (2H, d, J=2.7 Hz), 6.11 (lH, s), 7.14 (2H, d, J=8.2 Hz), 7.32 (2H, d, J=8.2 Hz)

Mass (APC1): 258.33 (M+H)⁺

(11) 4-[4-(4-(Trifluoromethyl)phenyl)3,6-dihydro-1(2H)-pyridyl]butanimidamide

¹H NMR (DMSO-d₆, δ): 1.70-2.00 (2H, m), 2.20-3.80 (10H, m), 6.35 (1H, s), 7.50-7.90 (4H, m), 8.53 (3H, brs)

Mass (ESI): 312.3 (M+H)⁺

(12) 4-[4-(4-Methoxyphenyl)-3,6-dihydro-1(2H)-pyridyl]butanmidamide

¹H NMR (DMSO-d₆, δ): 1.70-2.00 (2H, m), 2.30-2.80 (8H, m), 3.06 (2H, d, J=3.0 Hz), 3.74 (3H, s), 6.04 (1H, s), 6.90 (2H, d, J=8.8 Hz), 7.36 (2H, d, J=8.8 Hz)

Mass (APCI): 274.27 (M+H)⁺

(13) 4-[4-(4-Chlorophenyl)-1-piperazinyl]butanimidamide

¹H NMR (DMSO-d₆, δ): 1.70-2.00 (2H, m), 2.20-2.70 (8H, m), 2.90-3.30 (4H, m), 6.94 (2H, d, J=9.1 Hz), 7.23 (2H, d, J=9.1 Hz), 8.97 (3H, brs)

Mass (APCI): 281.20 (M+H)⁺

(14) 4-[4-(4-Fluorophenyl)-1-piperazinyl]butanimidamide

¹H NMR (DMSO-d₆, δ): 1.70-2.00 (2H, m), 2.20-2.80 (8H, m), 3.05 (4H, t, J=5.0 Hz), 6.80-7.20 (4H, m), 8.80 (3H, brs)

Mass (ESI): 265.4 (M+H)⁺

(15) 4-[4-(4-Nitrophenyl)-1-piperazinyl]butanimidamide

¹H NMR (DMSO-d₆, δ): 1.70-4.00 (14H, m), 7.02 (2H, d, J=9.4 Hz), 8.06 (2H, d, J=9.4 Hz)

Mass (ESI): 292.4 (M+H)⁺

Preparation 25

To a solution of 4-(4-phenyl-3,6-dihydro-1(2H)-pyridyl)butanenitrile (0.75 g) in toluene was added dropwise 1N diisobutylaluninium hydride in hexane (6.63 ml) at −78° C., and the mixture was warmed up to 0° C. The reaction was quenched with 1N aqueous hydrochloric acid, basified with saturated aqueous sodium hydrogen carbonate. The mixture was filtered through celite and the filter cake was washed with dichloromethane, then the combined filtrate was dried over magnesium sulfate and concentrated. The residue was chromatographed on silica gel (80% ethyl acetate in hexane to 10% methanol in dichloromethane) to give 4-(4-Phenyl-3,6-dihydro-1(2H)-pyridyl)butanal (0.4 g) as an oil.

¹H NMR (CDCl₃, δ): 1.90-2.30 (2H, m), 2.40-2.60 (2H, m), 2.70-2.85 (2H, m), 2.85-3.10 (2H, m), 3.50-3.70 (2H, m), 6.04 (1H, m), 7.10-7.60 (5H, m)

Mass (APCI): 230.27 (M+H)⁺

Preparation 26

A slurry of 4-benzyloxybutanal, (3-oxo-1,3-dihydro-2-benzofuran-1-yl)-(triphenyl)phosphonium bromide (560 mg) and triethylamine (7.39 ml) in tetrahydroflran (50 ml) was stirred at room temperature overnight. The resulting precipitates were removed by filtration and washed with ethyl acetate, and then the combined filtrate was concentrated. The residue was chromatographed on silica gel using toluene as an eluent to give an oil, which was dissolved in ethanol and refluxed in the presence of hydrazine monohydrate (1.4 g) for 1 hour. The mixture was concentrated, then dichloromethane and water were added and the organic layer was separated. The aqueous layer was further extracted with dichloromethane, and then the combined extracts were dried over magnesium sulfate and concentrated. The residue was triturated with dichloromethane and diisopropylether, and then the resulting powder was collected, washed with diisopropylether and dried in vacuo to give 4-[4-(Benzyloxy)butyl]-1(2H)-phthalazinone (2.78 g).

¹H NMR (DMSO-d₆, δ): 1.50-2.00 (4H, m), 2.94 (2H, t, J=7.2 Hz), 3.49 (2H, t, J=6.1 Hz), 4.45 (2H, s), 7.10-7.50 (5H, m), 7.70-8.20 (3H, m), 8.26 (1H, dd, J=1.9, 7.1 Hz), 12.45 (1H, brs)

Mass (ESI): 309.3 (M+H)⁺

Preparation 27

To slurry of 4-[4-(benzyloxy)butyl]-1(2H)-phthalazinone in dichloromethane (5 ml) was added dropwise 1M boron tribromide in dichloromethane (0.97 ml), and the mixture was stirred at room temperature for 2 hours. The reaction was quenched with water and extracted with dichloromethane twice. The combined extracts were dried over magnesium sulfate and concentrated. The residue was triturated with diisopropylether, and the resulting powder was collected, washed with diisopropylether and dried in vacuo to give 4-(4-Bromobutyl)-1(2H)-phthalazinone.

¹H NMR (DMSO-d₆, δ): 1.70-2.10 (4H, m), 2.96 (2H, t, J=7.3 Hz), 3.61 (2H, t, J=6.4 Hz), 7.70-8.10 (3H, m), 8.27 (1H, d, J=8.2 Hz), 12.47 (1H, brs)

Mass (ESI): 305.0 (M+Na)³⁰

The following compounds were prepared in a similar manner to that of Preparation 26.

Preparation 28

(1) 4-[5-(Benzyloxy)pentyl]-1(2H)-phthalazinone

¹H NMR (DMSO-d₆, δ): 1.40-2.00 (6H, m), 2.80-3.70 (4H, m), 4.32 (2H, s), 7.20-7.50 (5H, m), 7.70-8.10 (3H, m), 8.27 (1H, d, J=7.4 Hz), 12.44 (1H, brs)

Mass (ESI): 345.3 (M+Na)³⁰

Preparation 29

(1) 4-(5-Bromopentyl)-1(2H)-phthalazinone

¹H NMR (DMSO-d₆, δ): 1.30-2.00 (6H, m), 2.93 (2H, t, J=7.5 Hz), 3.54 (2H, t, J=6.7 Hz), 7.70-8.20 (3H, m), 8.27 (1H, d, J=7.3 Hz), 12.45 (1H, brs)

Mass (ESI): 317.1 (M+Na)³⁰

Preparation 30

50% Pd/C catalyst (50% wet, 400 mg) was added to a solution of 4-(4-biphenylyl)-1,2,3,6-etrahydropyridine (470mg) in a mixture of tetrahydrofuran (10 ml), methanol (20 ml) and acetic acid (10 ml). The mixture was stirred under hydrogen at atmospheric pressure until gas absorption ceased. After filtration through celite and 25 removal of solvent, the residue was dissolved in a mixture of ethyl acetate and aqueous sodium hydrogen carbonate. The aqueous phase was separated and the organic phase was washed with brine and dried over magnesium sulfate. Evaporation of the solvent afforded 4-(4-biphenylyl)piperidine (432mg).

Mass: 238.1 (M+H)⁺

Preparation 31

To a solution of 4-(4-fluorophenyl)-3,6-dihydro-1(2H)-pyridine (1 g) and ethyl 4-oxopentanoate (0.961 ml) in toluene was added a catalytic amount of p-toluenesulfonic acid (54 mg), and the mixture was stirred under reflux to remove librated water azeotropically. After stirring for 3 hours, the mixture was cooled and diluted with dichloroethane. To the mixture were added sodium tri(acetoxy)borohydride (3.59 g) and acetic acid (0.97 ml) in sequence, and the mixture was stirred at room temperature for 1 hour. The mixture was diluted with water, neutralized and extracted with dichloromethane three times. The combined extracts were dried over magnesium sulfate and concentrated. The residue was chromatographed on silica gel using ethyl acetate as an eluent to give Ethyl 4-[4-(4-fluorophenyl)-3,6-dihydro-1(2H)-pyridyl]-4-mehtylbutanoate (0.72 g)

¹H NMR (DMSO-d₆, δ): 0.80-4.30(19H, m), 6.13(1H, m), 6.80-7.60(4H, m).

Mass(ESI): 306.3 (M+H)⁺

Preparation 32

A mixture of 4-[4-(trifluoromethyl)phenyl]piperidine hydrochloride (1.18 g), 4-bromobutyronitrile (0.662 ml) and triethylamine (1.86 ml) in N,N-dimethylformamide (20 ml) was stirred at 80° C. overnight. The mixture was diluted with water, extracted with ethyl acetate twice. The combined extracts were washed with water three times, dried over magnesium sulfate and concentrated. The residue was dissolved in ethyl acetate and treated with silica gel (10 g). Silica gel was removed by filtration and washed with ethyl acetate . The combined filtrate was concentrated to give 4-[4-[4-(trifluoromethyl)phenyl]piperidino]butanenitrile as an oil.

¹H NMR (DMSO-d₆, δ): 1.40-3.20(15H, m),7.48(2H, d, J=8.2 Hz), 7.65(2H, d).

Mass(ESI): 297.2 (M+H)⁺

The following compound was obtained according to a similar manner to that of Preparation 32.

Preparation 33

4-[4-[4-(Trifluoromethoxy)phenyl]-3,6-dihydro-1(2H)-pyridyl]butanenitrile

¹H NMR (DMSO-d₆, δ): 1.60-3.30(12H, m), 6.20(1H, m), 7.00-7.80(4H, m).

Mass(ESI): 311.2 (M+H)⁺

Preparation 34

Under a nitrogen atmosphere, 4-bromobutanenitrile (402 mg) and triethylamine (0.76ml) was added successively to a suspension of 4-(4-biphenylyl)piperidine (430 mg) in N,N-dimethylformamide (5 ml) at room temperature. The mixture was stirred for 15 hours at 80° C. and cooled to room temperature. The mixture was poured into a mixture of water and chloroform and the aqueous layer was separated. The organic layer was washed with brine and dried over magnesium sulfate. The solvent was evaporated and the residue was purified by column chromatography on silica gel eluting with dichloromethane-acetone to afford 4-[4-(4-biphenylyl)piperidino]butanenitrile (411 mg).

Mass: 305.2 (M+H)⁺

The following compounds [Preparations 35 and 36] were obtained according to a similar manner to that of Preparation 34.

Preparation 35

4-[4-(3,4-dichlorophenyl)-1-piperazinyl]butanenitrile

¹H NMR (CDCl₃, δ): 1.85(2H, m), 2.4-2.7(8H, m), 3.1-3.2(4H, m), 6.72(1H, dd, J=9.0, 3.0 Hz), 6.95(1H, d, J=3.0 Hz), 7.28(1H, d, J=9.0 Hz).

Mass: 303.2 (M+Na)³⁰

Preparation 36

4-[4-(4-biphenylyl)-1,2,3,6-tetrahydropyridyl]butanenitrile

Mass: 303.2 (M+H)⁺

Preparation 37

To a suspension of ammonium chloride (1.09 g) in toluene (20 ml) was added dropwise 2N trimethylaluminium in toluene (10.2 ml) at 0° C. under nitrogen, and the mixture was stirred at room temperature for 2 hours. To this aluminum amide reagent was added dropwise 4-[4-[4-(trifluoromethyl)phenyl]piperidino]butanenitrile (1.21 g) in toluene (20 ml) at room temperature, and this solution was stirred at 80° C. overnight. The reaction mixture was carefully poured into a suspension of silica gel (15 g) in chloroform (40 ml). Silica gel was removed by filtaution and washed with methanol (50 ml) twice, and the combined filtrate was concentrated. The residue was chromatographed on alumina (30 g) (methanol/dichloromethane=1/4) to give 4-[4-[4(trifluoromethyl)phenyl]piperidino]butanamidine (1.40 g) as an oil.

¹H NMR (DMSO-d₆, δ):1.30-3.80(15H, m), 7.49(2H, d, J=7.9 Hz), 7.70(2H, d, J=7.9 Hz), 8.75(3H, brs).

Mass(ESI): 314.4 (M+H)⁺

The following compounds [Preparation 38 to 42] were obtained according to a similar manner to that of Preparation 37.

Preparation 38

4-[4-(4-Fluorophenyl)-3,6-dihydro-1(2H)-pyridyl]-4-methylbutanamidine

¹H NMR (DMSO-d₆, δ): 0.80-4.40(14H, m), 6.15(1H, m), 6.90-7.70(4H, m), 8.80(3H, brs).

Mass(ESI): 276.2 (M+H)⁺

Preparation 39

4-[4-[4-(Trifluoromethoxy)phenyl]-3,6-dihydro-1(2H)-pyridyl]butanamidine

¹H NMR (DMSO-d₆, δ): 1.50-4.00(12H, m), 6.22(1H, m), 7.32(2H, d, J=8.2 Hz), 7.56(2H, d, J=8.2 Hz).

Mass(ESI): 328.3 (M+H)⁺

Preparation 40

To a solution of 4-(4-fluorophenyl)-3,6-dihydro-1(2H)-pyridine (1 g) and ethyl 4-oxopentanoate (0.961 ml) in toluene was added a catalytic amount of p-toluenesulfonic acid (54 mg), and the mixture was stirred under reflux to remove librated water azeotropically. After stirring for 3 hours, the mixture was cooled and diluted with dichloroethane. To the mixture were added sodium tri(acetoxy)borohydride (3.59 g) and acetic acid (0.97 ml) in sequence, and the mixture was stirred at room temperature for 1 hour. The mixture was diluted with water, neutralized and extracted with dichloromethane three times. The combined extracts were dried over magnesium sulfate and concentrated. The residue was chromatographed on silica gel using ethyl acetate as an eluent to give Ethyl 4-[4-(4-fluorophenyl)-3,6-dihydro-1(2H)-pyridyl]pentanoate (0.72 g)

¹H NMR (DMSO-d₆, δ): 0.80-4.30(19H, m), 6.13(1H, m), 6.80-7.60(4H, m).

Mass(ESI): 306.3 (N+H)⁺

Preparation 41

4-[4-(3,4-dichlorophenyl)-1-piperazinyl]butanamidine

¹HNMR (DMSO-d₆, δ): 1.6-1.9(2H, m), 2.2-2.6(8H, m), 3.1-3.3(4H, m), 6.94(1H, dd, J=9.0, 2.5 Hz), 7.15(1H, d, J=2.5 Hz), 7.39(1H, d, J=9.0 Hz).

Mass: 316.2 (M+H)⁺

Preparation 42

4-[4-(4-biphenylyl)-1,2,3,6-tetrahydropyridyl]butanamidine

Mass: 320.1 (M+H)⁺

### EXAMPLE 1

A suspension of 4-(4-phenyl-3,6-dihydro-1(2H)-pyridyl)butanimidamide (107 mg), cyclohexanone-2-carboxylic acid ethyl ester (50 mg), potassium carbonate (568 mg) in ethanol (5 ml) was stirred at 80° C. overnight. The mixture was diluted with water and extracted with dichloromethane twice. The combined extracts were dried over magnesium sulfate and concentrated. The residue was purified by preparative thin layer chromatography on silica gel using 10% methanol in dichloromethane as an eluent to give 2-[3-(4-Phenyl-3,6-dihydro-1(2H)-pyridyl)propyl]-5,6,7,8-tetrahydro-4(3H)-quinazolinone (58 mg) as a colerless powder.

¹H NMR (CDCl₃, δ): 1.40-2.20 (6H, m), 2.30-3.00 (10H, m), 3.10-3.40 (2H, m), 6.10 (1H, s), 7.10-7.60 (5H, m)

Mass (APCI): 350.20 (M+H)⁺

### EXAMPLE 2

The following compounds were prepared in a similar manner to that of Example 1.

(1) 2-{3-[4-(4-Chlorophenyl)-2,2-dimethyl-1-piperazinyl]propyl}5,6,7,8-tetrahydro-4(3H)-quinazolinone

¹H NMR (DMSO-d₆, δ): 1.02 (6H, s), 1.40-1.90 (6H, m), 2.10-2.70 (10H, m), 2.83 (2H, s), 2.90-3.20 (2H, m), 6.89 (2H, d, J=9.0 Hz), 7.20 (2H, d, J=9.0 Hz), 12.28 (1H, brs)

Mass (ESI): 415.4 (M+H)⁺

(2) 2-{3-[(2S)4-(4-Chlorophenyl)-2-methyl-1-piperazinyl]propyl}-5,6,7,8-tetrahydro-4(3H)-quinazolinone

¹H NMR (DMSO-d₆, δ): 1.02 (3H, d, J=5.3 Hz), 1.40-3.60 (21H, m), 6.91 (2H, d, J=9.1 Hz), 7.20 (2H, d, J=9.1 Hz), 12.18 (1H, brs)

Mass (ESI): 401.2 (M+H)⁺

(3) 2-{3-[4-(4-Chlorophenyl)-3,3-dimethyl-1-piperazinyl]propyl}-5,6,7,8-tetrahydro-4(3H)-quinazolinone

¹H NMR (DMSO-d₆, δ): 0.96 (6H, s), 1.50-2.00 (2H, m), 2.00-3.20 (14H, m), 7.07 (2H, d, J=8.7 Hz), 7.29 (2H, d, J=8.7 Hz), 12.13 (1H, brs)

Mass (ESI): 415.4 (M+H)⁺

(4) 2-{3-[(2R,6S)-4-(4-Chlorophenyl)-2,6-dimethyl-1-piperazinyl]propyl}-5,6,7,8-tetrahydro-4(3H)-quinazolinone

¹H NMR (DMSO-d₆, δ): 1.06 (6H, d, J=6.0 Hz), 1.40-1.90 (6H, m), 2.10-3.80 (12H, m), 6.92 (2H, d, J=9.0 Hz), 7.20 (2H, d, J=9.0 Hz), 12.18 (1H, brs)

Mass (ESI): 415.4 (M+H)⁺

(5) 2-{3-[(2R,6R)-4-(4-Chlorophenyl)-2,6-dimethyl-1-piperazinyl]propyl)-5,6,7,8-tetrahydro-4(3H)-quinazolinone

¹H NMR (DMSO-d₆, δ): 0.99 (6H, d, J=6.1 Hz), 1.40-2.00 (6H, m), 2.10-3.30 (14H, m), 6.90 (2H, d, J=8.9 Hz), 7.20 (2H, d, J=8.9 Hz), 12.18 (1H, brs)

Mass (ESI): 415.4 (M+H)⁺

(6) 2-{3-[(2S,6S)-4-(4-Chlorophenyl)-2,6-dimethyl-1-piperazinyl]propyl}-5,6,7,8-tetrahydro-4(3H)-quinazolinone

¹H NMR (DMSO-d₆, δ): 0.99 (6H, d, J=6.1 Hz), 1.40-2.00 (6H, m), 2.10-3.30 (14H, m), 6.90 (2H, d, J=8.9 Hz), 7.20 (2H, d, J=8.9 Hz), 12.18 (1H, brs)

Mass (ESI): 415.4 (M+H)⁺

(7) 2-{3-[4-(4-Fluorophenyl)-3,6-dihydro-1(2H)-pyridyl]propyl}-5,6,7,8-tetrahydro-4(3H)-quinazolinone

¹H NMR (DMSO-d₆, δ): 1.40-2.00 (6H, m), 2.10-2.70 (12H, m), 3.04 (2H, d, J=2.6 Hz), 6.09 (1H, s), 7.00-7.60 (4H, m), 12.11 (1H, brs)

Mass (APCI): 368.20 (M+H)⁺

(8) 2-{3-[4*4-Chlorophenyl)-3,6-dihydro-1(2H)-pyridyl]propyl}-5,6,7,8-tetrahydro-4(3H)-quinazolinone

¹H NMR (DMSO-d₆, δ): 1.40-2.00 (6H, m), 2.20-2.80 (12H, m), 3.04 (2H, d, J=3.0 Hz), 6.17 (11H, s), 7.20-7.60 (4H, m), 12.11 (1H, brs)

Mass (ESI): 384.3 (M+H)⁺

(9) 2-{3-[4-(4-Methylphenyl)-3,6-dhydro-1(2H)-pyridyl]propyl}-5,6,7,8-tetrahydro-4(3H)-quinazolinone

¹H NMR (DMSO-d₆, δ): 1.40-2.00 (6H, m), 2.10-2.80 (15H, m), 3.04 (2H, m), 6.07 (1H, s), 7.12 (2H, d, J=8.0 Hz), 7.30 (2H, d, J=8.0 Hz), 12.09 (1H, brs)

Mass (ESI): 364.4 (M+H)⁺

(10) 2-{3-[4-(4-(Trifluoromethyl)phenyl)-3,6-dihydro-1(2H)pyridyl]propyl}-5,6,7,8-tetrahydro-4(3H)-quinazolinone

¹H NMR (DMSO-d₆, δ): 1.45-1.75 (4H, m), 1.80-2.00 (2H, m), 2.10-2.80 (12H, m), 3.08 (2H, d, J=1.4 Hz), 6.31 (1H, s), 7.50-7.80 (4H, m)

Mass (ESI): 418.3 (M+H)⁺

(11) 2-{3-[4-(4Methoxyphenyl)-3,6Aihydro-1(2H)-pyridyl]propyl}-5,6,7,8-tetrahydro-4(3H)-quinazolinone

¹H NMR (DMSO-d₆, δ): 1.40-2.00 (6H, m), 2.10-3.20 (12H, m), 3.74 (3H, s), 6.00 (1H, s), 6.88 (2H, d, J=8.8 Hz), 7.34 (2H, d, J=8.8 Hz), 12.08 (1H, brs)

Mass (APCI): 380.20 (M+H)⁺

(12) 2-{3-[4-(4-Chlorophenyl)-1-piperazinyl]propyl)-5,6,7,8-tetrahydro-4(3H)-quinazolinone

¹H NMR (DMSO-d₆, δ): 1.40-2.00 (6H, m), 2.00-3.70 (16H, m), 6.91 (2H, d, J=9.0 Hz), 7.21 (2H, d, J=9.0 Hz), 12.17 (1H, brs)

Mass (APCI): 387.07 (M+H)⁺

(13) 2-{3-[4-(4-Fluorophenyl)-1-piperazinyl]propyl}-5,6,7,8-tetrahydro-4(3H)-quinazolinone

¹H NMR (DMSO-d₆, δ): 1.40-2.00 (6H, m), 2.10-3.20 (16H, m), 6.80-7.20 (4H, m), 12.16 (1H, brs)

Mass (APCI): 371.07 (M+H)⁺

### EXAMPLE 3

The following compounds were prepared in a similar manner to that of Example 1.

(1) 2-{3-[4-(3,4-Difluorophenyl)3,6-dihydro-1(2H)-pyridyl]propyl)-3,5,7,8-tetrahydro-4H-thiopyrano[4,3-d]pyrimidin-4-one

¹H NMR (DMSO-d₆, δ): 1.70-2.00 (2H, m), 2.30-3.00 (12H, m), 3.07 (2H, d, J=2.9 Hz), 3.40 (2H, s), 6.02 (1H, s), 7.00-7.60 (3H, m), 12.35 (1H, brs)

Mass (ESI): 404.2 (M+H)⁺

(2) 2-{3-[4-(4-Fluorophenyl)-3,6-dihydro-1(2H)-pyridyl)propyl}-3,5,7,8-tetrahydro-4H-thiopyrano[4,3]pyrimidin-4-one

¹H NMR (DMSO-d₆, δ): 1.70-2.00 (2H, m), 2.20-2.80 (4H, m), 3.04 (2H, d, J=2.8 Hz), 6.09 (1H, s), 7.00-7.60 (4H, m), 12.34 (1H, brs)

Mass (APCI): 386.00 (M+H)⁺

(3) 2-{3-[4-(4-Chlorophenyl)-3,6-dihydro-1(2H)-pyridyl]propyl)-3,5,7,8-tetrahydro-4H-thiopyrano[4,3-d]pyrimidin-4-one

¹H NMR (DMSO-d₆, δ): 1.70-2.00 (2H, m), 2.20-3.20 (16H, m), 6.17 (1H, s), 7.20-7.60 (4H, m), 12.35 (1H, brs)

Mass (APCI): 401.93 (M+H)⁺

(4) 2-{3-[4-(4-Methylphenyl)-3,6-dihydro-1(2H)-pyridyl]propyl}-3,5,7,8-tetrahydro-4H-thiopyrano[4,3-d]pyrimidin-4-one

¹H NMR (DMSO-d₆, δ): 1.70-2.00 (2H, m), 2.27 (3H, s), 2.30-2.90 (12H, m), 3.03 (2H, d, J=2.8 Hz), 3.38 (2H, s), 6.07 (1H, s), 7.12 (2H, d, J=8.2 Hz), 7.30 (2H, d, J=8.2 Hz), 12.35 (1H, brs)

Mass (APCI): 382.13 (M+H)⁺

(5) 2-{3-[4-(4-(Trifluoromethyl)phenyl)-3,6-dihydro-1(2H)-pyridyl]propyl}-3,5,7,8-tetrahydro-4H-thiopyrano[4,3-d]pyrimidin-4-one

¹H NMR (DMSO-d₆, δ): 1.70-2.00 (2H, m), 2.30-3.00 (14H, m), 3.08 (2H, d, J=2.5 Hz), 6.31 (1H, s), 7.40-7.80 (4H, m)

Mass (APCI): 434.33 (M−H)⁻

(6) 2-{3-[4-(4-Methoxyphenyl)-3,6-dihydro-1(2H)-pyridyl]propyl}-3,5,7,8-tetrahydro-4H-thiopyrano[4,3-d]pyrdin-4-one

¹H NMR (DMSO-d₆, δ): 1.70-2.00 (2H, m), 2.20-3.20 (14H, m), 3.38 (2H, s), 3.74 (3H, s), 6.00 (1H, s), 6.88 (2H, d, J=8.8 Hz), 7.34 (2H, d, J=8.8 Hz), 12.36 (1H, brs)

Mass (ESI): 398.3 (M+H)⁺

(7) 2-{3-[(4-Chlorophenyl)-1-piperazinyl]propyl} -3,5,7,8-tetrahydro-4H-thiopyrano[4,3-d]pyrimidin-4-one

¹H NMR (DMSO-d₆, δ): 1.70-2.00 (2H, m), 2.20-3.20 (16H, m), 3.40 (2H, s), 6.92 (2H, d, J=9.1 Hz), 7.21 (2H, d, J=9.1 Hz), 12.36 (1H, brs)

Mass (APCI): 405.3 (M+H)⁺

(8) 2-{3-[(4-Fluorophenyl)-1-piperazinyl]propyl} -3,5,7,8-tetrahydro-4H-thiopyrano[4,3-d]pyrimidin-4-one

¹H NMR (DMSO-d₆, δ): 1.70-2.00 (2H1, m), 2.20-2.90 (12H, m), 3.01 (4H, t, J=4.6 Hz), 3.40 (2H, s), 6.80-7.20 (4H, m), 12.43 (1H, brs)

Mass (APCI): 389.2 (+H)⁺

(9) 2-{3-[(4—Nitrophenyl)-1-piperazinyljpropyl}-3,5,7,8-tetrahydro-4H-thiopyrano[4,3-d]pyrimidin-4-one

¹H NMR (DMSO-d₆, δ): 1.70-2.00 (2H, m), 2.20-3.00 (16H, m), 3.40 (2H, s), 7.02 (2H, d, J=9.4 Hz), 8.05 (2H, d, J=9.4 Hz), 12.41 (1H, brs)

Mass (ESI): 416.2 (M+H)⁺

(10) 2-[3-(4-Phenyl-3,6-dihydro-1(2H)-pyridyl)propyl]-3,5,7,8-tetrahydro-4H-thiopyrano[4,3-d]pyrimidin-4-one

¹H NMR (DMSO-d₆, δ): 1.70-2.00 (2H, m), 2.20-3.90 (16H, m), 6.12 (1H, s), 7.10-7.60 (5H, m), 12.38 (1H, brs)

Mass (APCI): 368.07 (M+H)⁺

(11) 2-[3-(4-Phenyl-3,6-dihydro-1(2H)-pyridyl)propyl]-5,6,7,8-tetrahydropyrido[4,3-d]pyrimidin-4(3H)-one

¹H NMR (DMSO-d₆, δ): 1.70-2.00 (2H, m), 2.20-3.80 (16H, m), 6.15 (1H, s), 7.00-7.60(5H, m)

Mass (ESI): 351.3 (M+H)⁺

(12) 2-[3-(4-Phenyl-3,6-dihydro-1(2H)-pyridyl)propyl]-3,5,7,8-tetrahydro-4H-pyrano[4,3d]pyrimidin-4-one

¹H NMR (DMSO-d₆, δ): 1.70-2.00 (2H, m), 2.20-2.80 (1OH, m), 3.07 (2H, s) 3.80 (2H, t, J=5.5 Hz), 4.29 (2H, s), 6.12 (1H, s), 7.10-7.70 (5H, m)

Mass (APCI): 352.2 (M+1)+

### EXAMPLE 4

The following compounds were prepared in a similar manner to that of Example 1.

(1) 2-[3-(4-Phenyl-3,6dihydro-1(2H)-pyridyl)propyl]-3,5,6,7-tetrahydro-4H-cyclopenta[d]pyrimidin-4-one

¹H NMR (CDCl₃, δ): 1.70-2.30 (4H, m), 2.40-3.40 (14H, m), 6.06 (1H, s), 7.00-7.60 (5H, m)

Mass (APCI): 336.20 (M+H)⁺

(2) 2-[3-(4-Phenyl-3,6-dihydro-1(2H)-pyridyl)propyl]-3,5,6,7,8,9-hexahydro-4H-cyclohepta[d)pyrimidin-4-one

¹H NMR (CDCl₃, δ): 1.00-2.40 (8H, m), 2.40-3.40 (14H, m), 6.07 (1H, s), 7.00 -7.60(5H, m)

Mass (APCI): 364.20 (M+H)⁺

(3) 2-{3-[4-(4-Fluorophenyl)-3,6-dihydro-1(2H)-pyridyl]propyl}-7,8-dihydro-3H-thiopyrano[3,2-d]pyrimidin-4(6H)-one

¹H NMR (DMSO-d₆, δ): 1.70-2.20 (2H, m), 2.30-3.20 (14H, m), 6.10 (1H, s), 7.00-7.60 (4H, m), 12.37 (1H, brs)

Mass (ESI): 386.2 (M+H)⁺

### EXAMPLE 5

A mixture of 4-[4-(4-fluorophenyl)-3,6-dihydro-1(2H)-pyridyl]butanimidamide (90 mg) and 2H-pyrido-[2,3-d][1,3]oxazine-2,4(1H)-dione (79 mg) in pyridine (5 ml) was stirred at 120° C. overnight. The mixture was concentrated and coevaporated with toluene twice. The residue was purified by preparative thin layer chromatography using 10% methanol in dichloromethane as an eluent to give 2-{3-[4-(4-Fluorophenyl)-3,6-dihydro-1(2H)-pyridyl]propyl}-pyrido[2,3-d]pyrimidin-4(3H)-one, which was converted to the corresponding hydrochloride salt (40 mg) by treatment of 4N hydrogen chloride in ethyl acetate.

¹H NMR (DMSO-d₆, δ): 2.00-5.30 (12H, m), 6.18 (1H, s), 7.00-7.80 (5H, m), 8.55 (1H, dd, J=2.0, 8.0 Hz), 8.93 (1H, dd, J=2.0, 4.7 Hz)

Mass (ESI): 365.5 (M+H)⁺

### EXAMPLE 6

To a solution of 2-[3-(4-phenyl-3,6-dihydro-1(2H)-pyridyl)propyl]-5,6,7,8-tetrahydropyrido[4,3-d]pyrimidin-4(3H)-one (28 mg) in dichmoromethane (5 ml) and methanol (1 ml) were added 37% aqueous formaldehyde (0.063 ml) and sodium triacetoxyborohydride (51 mg) in sequence, then the mixture was stirred at room temperature for 2 hours. The reaction was quenched with silica gel (0.2 g) and concentrated. The residue was chromatographed on silica gel (20% methanol in dichloromethane) to give 6-Methyl-2-[3-(4-phenyl-3,6dihydro-1(2H)-pyridyl)propyl]-5,6,7,8-tetrahydropyrido[4,3-d]pyrimidin-4(3H)-one the objective compound as a brown powder.

¹H NMR (DMSO-d₆, δ): 1.70-3.20 (21H, m), 6.11 (1H, s), 7.00-7.50 (5H, m), 12.26 (1H, brs)

Mass (ESI): 365.4 (M+H)⁺

### EXAMPLE 7

A suspension of 4-(4-phenyl-3,6-dihydro-1(2H)-pyridyl)butanal (0.18 g), (3-oxo-1,3-dihydro-2-benzofuran-1-yl)(triphenyl)phosphonium bromide (560 mg) and triethylamine (0.328 ml) in tetrahydrofuran (20 ml) was stirred at room temperature for 3 hours. The reaction was quenched with water and extracted with ethyl acetate twice. The combined extracts were dried over magnesium sulfate and concentrated. The residue was chromatographed on silica gel using ethyl acetate as an eluent to give oil, which was dissolved in ethanol and refluxed in the presence of hydrazine monohydrate (77 mg) for 1 hour. The mixture was concentrated, then dichloromethane and water was added and the organic layer was separated. The aqueous layer was further extracted with dichloromethane, then the combined extracts were dried over magnesium sulfate and concentrated. The residue was chromatographed on silica gel (ethyl acetate to 5% methanol in dichloromethane), and then the fractions eluted with 5% methanol in dichloromethane were combined and concentrated. The residue was triturated with a mixture of ethyl acetate and diisopropyl ether to give 4-[4-(4-Phenyl-3,6dihydro-1(2H)-pyridyl)butyl]-1(2H)-phthalazinone (46 mg) as a pale yellow powder.

¹H NMR (DMSO-d₆, δ): 1.10-1.90 (4H, m), 2.30-3.00 (8H, m), 3.07 (2H, d, J=2.8 Hz), 6.15 (1H, s), 7.10-8.40 (9H, m), 12.45 (1H, brs)

Mass (APCI): 360.07 (M+H)⁺

### EXAMPLE 8

A mixture of 4-(4-Bromobutyl)-1(2H)-phthalazinone (100 mg), 4-fluorophenyl-1,2,5,6-tetrahydropyridine hydrochloride (91 mg) and triethylamine (0.149 ml) in N,N-dimethylformamide (5 ml) was stirred at room temperature overnight. The mixture was diluted with water and extracted with ethyl acetate twice. The combined extracts were washed with water three times, dried over magnesium sulfate and concentrated. The residue was purified by preparative thin layer chromatography (10% methanol in dichloromethane) to give 4-{4-[4-(4-Fluorophenyl)-3,6-dihydro-1(2H)pyridyl]butyl}-1(2H)-phthalazinone (70 mg) as a colorless powder.

¹H NMR (DMSO-d₆, δ): 1.40-2.00 (4H, m), 2.30-3.30 (1OH, m), 6.12 (1H, s), 7.00-7.60 (5H, m), 7.70-8.00 (2H, m), 8.04 (1H, d, J=7.6 Hz), 8.26 (1H, d, J=7.6 Hz), 12.44 (1H, brs)

Mass (ESI): 378.3 (M+H)⁺

### EXAMPLE 9

The following compounds were prepared in a similar manner to that of Example 8.

(1) 4-(4-[4-(4-Chlorophenyl)-3,6dihydro-1(2H-pyridyl]butyl}-1(2H)-phthalazinone

¹H NMR (DMSO-d₆, δ): 1.40-1.90 (4H, m), 2.30-2.80 (8H, m), 2.95 (2H, t, J=7.3 Hz), 3.06 (2H, d, J=2.5 Hz), 6.20 (1H, s), 7.20-7.60 (5H, m), 7.70-8.00 (2H, m), 8.04 (1H, dd, J=1.5, 7.6 Hz), 8.26 (1H, dd, J=1.5, 7.6 Hz), 12.45 (1H, brs)

Mass (ESI): 394.2 (M+H)⁺

(2) 4-{4-[4-(4-Methylphenyl)-3,6Aihydro (2H)-pyridyl]butyl}-1(2H)-phthalazinone

¹H NMR (DMSO-d₆, δ): 1.40-1.90 (4H, m), 2.28 (3H, s), 2.30-3.30 (10H, m), 6.10 (1H, s), 7.13 (2H, d, J=8.1 Hz), 7.31 (2H, d, J=8.1 Hz), 7.70-8.00 (2H,m), 8.05 (1H, d, J=7.4 Hz), 8.26 (1H, d, J=7.4 Hz), 12.45 (1H, brs)

Mass (ESI): 374.4 (M+H)⁺

(3) 4-{4-[4-(4-(Trifluoromethyl)phenyl)-3,6-dihydro-1(2H)-pyridyl]butyl}-1(2H)-phthalazinone

¹H NMR (DMSO-d₆, δ): 1.40-2.00 (4H, m),2.30-3.30 (10H, m), 6.34 (1H, s), 7.60-8.00 (6H, m), 8.04 (1H, d, J=7.7 Hz), 8.26 (1H, d, J=7.7 Hz), 12.45 (1H, brs)

Mass (ESI): 428.3(M+H)⁺

(4) 4-{4-[4-(4-Chlorophenyl)-1-piperazinyl]butyl}-1(2H)-phthalazinone

¹H NMR (DMSO-d₆, δ): 1.40-1.90 (4H, m), 2.20-3.70 (10H, m), 6.92 (2H, d, J=9.1 Hz), 7.21 (2H, d, J=9.1 Hz), 7.70-8.00 (2H, m), 8.04 (1H, d, J=7.4 Hz) 8.26 (1H, d, J=7.4 Hz), 12.45 (1H, brs)

Mass (ESI): 397.3 (M+H)⁺

(5) 4-{4-[4-(4-Fluorophenyl)-1-piperazinyl]butyl}-1(2H)-phthalazinone

¹H NMR (DMSO-d₆, δ): 1.40-1.90 (4H, m), 2.20-3.30 (12H, m), 6.80-7.20 (4H, m), 7.70-8.00 (2H, m), 8.04 (1H, dd, J=1.6, 7.6 Hz), 8.26 (1H, dd, J=1.6, 7.6 Hz), 12.45 (1H, brs)

Mass (ESI): 381.3 (M+H)⁺

(6) 4-{4-[4-(4—Nitrophenyl)-1-piperazinyl]butyl}-1(2H)-phthalazinone

¹H NMR (DMSO-d₆, δ): 1.40-2.00 (4H, m), 2.00-3.70 (12H, m), 7.02 (2H, d J=9.5 Hz), 7.70-8.20 (5H, m), 8.26 (1H, dd, J=l.1, 7.7 Hz), 12.45 (1H, brs)

Mass (ESI): 408.3 (M+H)⁺

(7) 4-[5-(4-Phenyl-3,6-dihydro-1(2H)-pyridyl)pentyl]-1(2H)-phthalazinone

¹H NMR (DMSO-d₆, δ): 1.20-2.00 (6H, m), 2.10-3.20 (1OH, m), 6.14 (1H, s), 7.10-7.60 (5H, m), 7.70-8.10 (3H, m), 8.26 (1H, d, J=7.4 Hz), 12.44 (1H, brs)

Mass (ESI): 374.4 (M+H)⁺

(8) 4-[4-(9-Methyl-1,3,4,9-tetrahydro-2H-pyrido[3,4-b]indol-2-yl)butyl]-1(2H)phthalazinone

¹H NMR (DMSO-d₆, δ): 1.40-2.00 (4H, m), 2.40-3.20 (8H, m), 3.59 (3H, s), 3.64 (2H, s), 6.80-7.20 (2H, m), 7.20-8.40 (4H, m), 12.56 (1H, brs)

Mass (ESI): 387.3 (M+H)⁺

### EXAMPLE 10

Oxalyl chloride (0.193 mL, 2.21 mmol) was dissolved in dichloromethane (3 mL) at −78° C. A solution of dimethylsulfoxide (0.392 mL, 5.52 mmol) in dichloromethane (1 mL) was added dropwise to that solution, and the mixture was stirred for 10 minutes at that temperature. A solution of 4-(4-hydroxybutyl)-1(2H)-isoquinolinone (60 mg, 0.276 mmol) in a mixed solvent of dichloromethane (1 mL) and dimethylsulfoxide (1 mL) was added dropwise. The mixture was stirred at −78° C. for 15 minutes, and at −45° C. for 40 minutes. Triethylamine (0.70 mL) was added dropwise, and the mixture was stirred at 0° C. for 1 hour. The crude product was used for next step without purification. The crude 4-(1-oxo-1,2-dihydro-4-isoquinolinyl)butanal (59 mg) was dissolved in dichloromethane (1 mL), and 4-phenyl-1,2,3,6-tetrahydropyridine (87.9 mg, 0.552 mmol) was added. Then sodium triacetoxyborohydride (117 mg, 0.552 mmol) and acetic acid (0.032 mL, 0.552 mmol) were added to the mixture, and it was stirred at room temperature for 15 hours. Purification over silica gel chromatography gave 5 4-[4-(4-phenyl-3,6fihydro-1(2H)-pyridyl)butyl]-1(2H)-isoquinolinone (24 mg, 24.2%) as product.

¹H NMR (200 MHz, DMSO-d₆, δ): 1.59 (4H, br s), 2.4-2.7 (8H, m), 3.06 (2H, d, J=2.9 Hz), 6.15 (1H, br s), 6.98 (1H, d, J=3.5 Hz), 7.1-7.6 (6H, m), 7.71 (1H, t, J=6.7 Hz), 7.78 (1H, d), 8.22 (1H, d, J=8.0 Hz), 11.09 (1H, br s)

### EXAMPLE 11

A suspension of 4-[4-(4-fluorophenyl)piperidino]butanamidine (97 mg), methyl 4-oxotetrahydrothiopyran-3-carboxylate (96 mg), potassium carbonate (509 mg) in ethanol (5 ml) was stirred at 80° C. overnight. The mixture was diluted with water and extracted with dichloromethane twice. The combined extracts were dried over magnesium sulfate and concentrated. The residue was purified by preparative thin layer chromatography on silica gel (methanoydichloromethane=1/9) to give 2-[3-[4-(4-Fluorophenyl)piperidino]propyl]-3,5,7,8-tetrahydro4H-tbiino[4,3-d]pyrimidin-4-one (55 mg) as a colerless powder.

¹H NMR (DMSO-d₆, δ): 1.00-3.70(21H, m), 6.90-7.40(4H, m), 12.64(1H, brs).

Mass(ESI): 388.3 (M+H)⁺

The following compounds [Example 12 to 27] were obtained according to a similar manner to that of Example 11.

### Example 12

2-[3-[4-(4-Methoxyphenyl)piperidino]propyl]-3,5,7,8-tetrahydro-4H-thiina[4,3-d]pyrimidin-4-one

¹H NMR (DMSO-d₆, δ): 1.00-3.60(21H, m), 3.71(3H, s),6.84(2H, d, J=8.7 Hz), 30 7.13(2H, d, J=8.7 Hz),12.47(1H, brs).

Mass(ESI): 400.3 (M+H)⁺

### EXAMPLE 13

2-[3-[4-(4-Methylphenyl)piperidino]propyl]-3,5,7,8-tetrahydro-4H-thiino[4,3-d]pyrimidin-4-one

¹H NMR (DMSO-d₆, δ): 1.30-3.70(24H, m), 6.90-7.20(4H, m), 12.61(1H, brs).

Mass(ESI): 384.2 (M+H)⁺

### EXAMPLE 14

2-{3-[4-(4-Fluorophenyl)piperidino]propyl} -5,6,7,8-tetrahydro-4(³H)-quinazolinone

¹H NMR (DMSO-d₆, δ): 1.30-3.20(23H, m),7.00-7.40(4H, m),12.38(1H, brs).

Mass(ESI): 370.3 (M+H)⁺

### EXAMPLE 15

2-[3-[4-(4-Chlorophenyl)piperidino]propyl]-5,6,7,8-tetrahydro-4(3H)-quinazolinone

¹H NMR (DMSO-d₆, δ): 1.30-3.20(23H, m), 7.10-7.60(4H, m), 12.36(1H, brs).

Mass(ESI): 386.4 (M+H)⁺

### EXAMPLE 16

2-[3-[4-(4-Methylphenyl)piperidino]propyl]-5,6,7,8-tetrahydro-4(3H)-quinazolinone

¹H NMR (DMSO-d₆, δ): 1.20-3.20(26H, m), 7.00-7.20(4H, m), 12.34(1H, brs).

Mass(ESI):366.4 (M+H)⁺

### EXAMPLE 17

2-[3-[4-(4-Methoxyphenyl)piperidino]propyl]-5,6,7,8-tetrahydro-4(3H)-quinazolinone

¹H NMR (DMSO-d₆, δ): 1.20-3.20(23H, m), 3.71(3H, s), 6.83(2H, d( J=8.6 Hz), 7.12(2H, d, J=8.6 Hz), 12.35(1H, brs).

Mass(ESI): 382.3 (M+H)⁺

### EXAMPLE 18

2-[3-[4-[4-(Trifluoromethyl)phenyl]piperidine]propyl]-3,5,7,8-tetrahydro-4H-thiino[4,3-dlpyrimidin-4-one

¹H NMR (DMSO-d₆, δ): 1.50-3.60(21H, m), 7.46(2H, d, J=8.2 Hz), 7.64(2H, d, J=8.2 Hz), 12.65(1H, brs).

Mass(ESI): 438.3 (M+H)⁺

### EXAMPLE 19

2-[3-[4-[4-(Trifluoromethyl)phenyl]piperidino]propyl]-5,6,7,8-tetrahydro-4(3H)-quinazolinone

¹H NMR (DMSO-d₆, δ): 1.30-3.20(23H, m), 7.45(2H, d, J=8.1 Hz), 7.64(2H, d, J=8.1 Hz), 12.36(1H, brs).

Mass(ESI): 420.3 (M+H)⁺

### EXAMPLE 20

2-[3-[(2R,6R)-4-Chlorophenyl-2,6-dimethyl-1-piperazinyl]propyl]-3,5,7,8-tetrahydro-4H-thiino[4,3-d]pyrimidin-4-one

¹HNMR (DMSO-d₆, δ): 0.99(6H, d, J=6.0 Hz), 1.50-3.70(18H, m), 6.90(2H, d, J=9.0 Hz), 7.20(2H, d, J=9.0 Hz), 12.45(1H, brs).

Mass(ESI): 433.1 (M+H)⁺

### EXAMPLE 21

2-[3-[4-(4-Fluorophenyl)-3,6-dihydro-1(2H)-pyridyl]-3-methylpropyl]-3,5,7,8-tetrahydro4H-thiino[4,3-d]pyrimidin-4-one hydrochloride

¹HNMR (DMSO-d₆, δ): 0.80-5.20(20H, m), 6.20(1H, m), 7.00-7.70(4H, m).

Mass(ESI): 400.1 (M+H)⁺

### EXAMPLE 22

2-[3-[4-[4-(Trifluoromethoxy)phenyl]-3,6-dihydro-1(2H)-pyridyl]propyl]-3,5,7,8-etrahydro-4H-thiino[4,3-d]pyrimidin-4-one

¹H NMR (DMSO-d₆, δ): 1.70-3.60(18H, m), 6.18(1H, m), 7.31(2H, d, J=8.1 Hz), 7.53(2H, d, J=8.1 Hz), 12.37(1H, brs).

Mass(ESI): 452.2 (M+H)⁺

### EXAMPLE 23

2-[3-[4-[4-(Trifluoromethoxy)phenyl]-3,6-dihydro-1(2H)-pyridyl]propyl]-5,6,7,8-tetrahydro-4(3H)-quinazolinone

¹H NMR (DMSO-d₆, δ): 1.40-3.20(20H, m), 6.18(1H, m), 7.31(2H, d, J=8.2 Hz), 7.53(2H, d, J=8.2 Hz), 12.13(1H, brs).

Mass(ESI): 434.2 (M+H)⁺

### EXAMPLE 24

2-[3-[4-(4-biphenylyl)piperidino]propyl]-3,5,7,8-tetrahydro-4H-thiino[4,3-d]pyrimidin-4-one

¹H NMR (DMSO-d₆, δ): 1.5-2.2(8H, m), 2.3-2.65(2H, m), 2.65-2.9(4H, m), 2.9-3.1(2H, m), 3.2-3.6(5H, m), 7.2-7.7(9H, m).

Mass: 446.4(M+H)⁺

### EXAMPLE 25

2-[3-[4-(3,4dichlorophenyl)-1-piperazinyl]propyl]-5,6,7,8-tetrahydro4(3H)-quinazolin

¹H NMR (DMSO-d₆, δ): 1.5-1.9(6H, m), 2.2-2.6(12H, m), 3.0-3.2(4H, m), 6.9(1H, dd, J=9.0, 2.8 Hz), 7.09(1H, d, J=2.8 Hz), 7.38(1H, d, J=9.0 Hz), 12.18 (1H, br, s).

Mass: 421.1, 423.2 (M+H)⁺

### EXAMPLE 26

2-[3-[4-(3,4-dichlorophenyl)-1-piperazinyl]propyl]-3,5,7,8-tetrahydro-4H-thiino[4,3-d]pyrimidin-4-one

¹H NMR (DMSO-d₆, δ): 1.7-1.85(2H, m), 2.2-2.6(8H, m), 2.7-2.9(4H, m), 3.0-3.2(4H, m), 3.39(2H, s), 6.90(1H, dd, J=9.0, 2.5 Hz), 7.10(1H, d, J=2.5 Hz), 7.37(1H, d, J=9 Hz), 12.4(1H, br s).

Mass: 441.1, 439.1(M+H)⁺

### EXAMPLE 27

2-[3-[4-(4-biphenylyl)-1,2,3,6-tetrahydropyridyl]propyl]-3,5,7,8-tetrahydro-4H-thiino[4,3-d]pyrimiidin-4-one

¹H NMR (DMSO-d₆, δ): 1.7-2.0(2H, m), 2.3-2.9(8H, m), 3.09(2H, s), 3.2-3.6(6H, m), 6.20(1H, s), 7.3-7.9(9H, m), 12.4(1H, s).

Mass: 444.2(M+H)⁺

1. A compound of the formula (I): 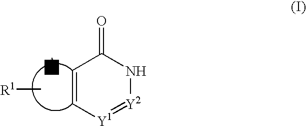
wherein
R¹ is hydrogen, halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from
the group consisting of nitrogen atom, oxygen atom, and sulfur atom, 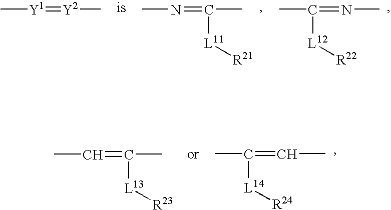
[wherein L¹¹, L¹², L¹³ and L¹⁴ is
(1) lower alkylene,
(2) lower alkenylene,
(3) cyclo(lower)alkylene,
(4) cyclo(lower)alkenylene,
(5) diradical of saturated- or unsaturated monocyclic group with one or more nitrogen atom(s), which is obtained after removal of one hydrogen atom from said monocyclic group, or
(6) N(R³)-L- (wherein R³ is hydrogen or lower alkyl, and L is lower alkylene or lower alkenylene), and
R²¹, R²², R²³and R²⁴is
(1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
(2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
(3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl],
provided that
when A and two adjacent carbon atoms of the six membered ring to be bonded with A form benzene ring,
then 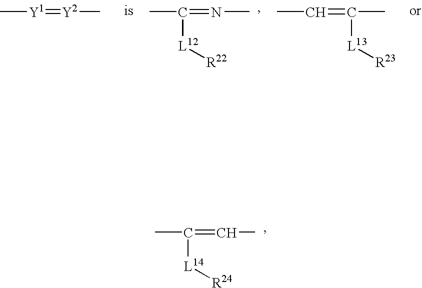
or its prodrug, or their salts.

2. The compound according to claim 1, wherein 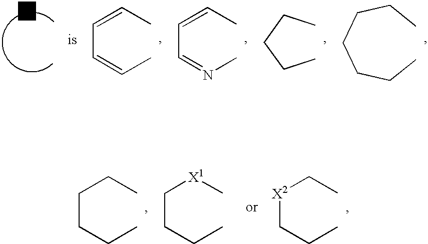
[wherein X¹ and X² is N, O or S].

3. The compound according to claim 2, wherein
R¹ is hydrogen, and
R²¹, R²², R²³ and R²⁴ is tetrahydropyridyl, piperidyl or piperazinyl, each of which is substituted with phenyl substituted with 1 or 2 substituent(s) selected from the group consisting of halogen, nitro, lower alkoxy, lower alkyl, halo(lower)alkyl, halo(lower)alkoxy and phenyl.

4. The compound according to claim 1, wherein
L¹¹ and L¹³ is lower alkylene.

5. The compound according to claim 1, wherein

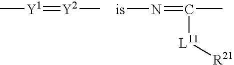

6. A pharmaceutically composition comprising a compound of the formula (I): 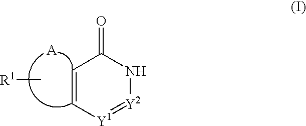
wherein
R¹ is hydrogen, halogen, lower alkyl or lower alkoxy,
A and two adjacent carbon atoms of the six membered ring to be bonded with A form benzene ring, pyridine ring, or five to seven membered partially saturated ring optionally containing one or more heteroatom(s) selected from
the group consisting of nitrogen atom, oxygen atom, and sulfur atom, 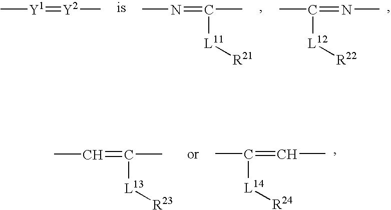
[wherein L¹¹, L¹², L¹³ and L¹⁴ is
(1) lower alkylene,
(2) lower alkenylene,
(3) cyclo(lower)alkylene,
(4) cyclo(lower)alkenylene,
(5) diradical of saturated- or unsaturated monocyclic group with one or more nitrogen atom(s), which is obtained after removal of one hydrogen atom from said monocyclic group, or
(6) N(R³)-L- (wherein R³ is hydrogen or lower alkyl, and L is lower alkylene or lower alkenylene), and
R²¹, R²², R²³ and R²⁴ is
(1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
(2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
(3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.],
provided that
when A and two adjacent carbon atoms of the six membered ring to be bonded with A form benzene ring,
then 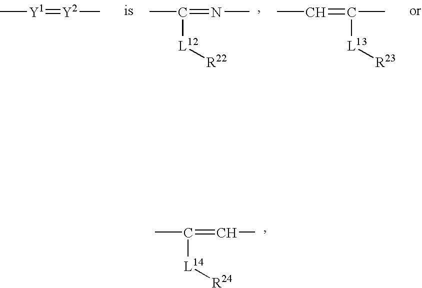
or its prodrug, or their pharmaceutically acceptable salts, and a pharmaceutically acceptable carrier, wherein said compound is present in an amount effective for inhibiting PARP activity.

7. The pharmaceutical composition of claim 6 for treating or preventing diseases ascribed by NMDA- and NO-induced toxicity.

8. The pharmaceutical composition of claim 6 for extending the lifespan or proliferative capacity of cells or altering gene expression of senescent cells

9. The pharmaceutical composition of claim 6 for treating or preventing tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Parkinson's disease; epilepsy; Amyotrophic Lateral Scleosis (ALS); Huntington's disease; schizophrenia; chronic pain; ischemia and nloss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; arteriosclerosis; osteoarthritis; osteoporosis; muscular dystrophy; degenerative diseases of skeletal muscle involving replicative senescence; age-related macular degeneration; immune senescence; AIDS; other immune senescence diseases; inflammatory bowel disorders (e.g., colitis); arthritis; diabetes; endotoxic shock; septic shock; or tumor.

10. A method of inhibiting PARP activity comprising administering a compound of the formula (I): 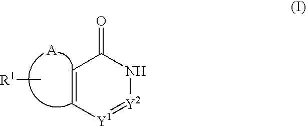
wherein
R¹ is hydrogen, halogen, lower alkyl and lower alkoxy,
A and two adjacent carbon atoms of the six membered ring to be bonded with A form benzene ring, pyridine ring, or five to seven membered partially saturated ring optionally containing one or more heteroatom(s) selected from the group consisting of nitrogen atom, oxygen atom, and sulfiir atom, 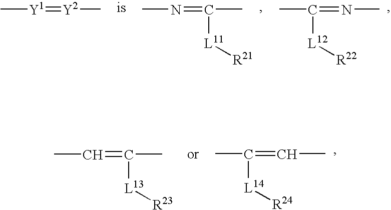
[wherein L¹¹, L¹², L¹³ and L¹⁴ is
(1) lower alkylene,
(2) lower alkenylene,
(3) cyclo(lower)alkylene,
(4) cyclo(lower)alkenylene,
(5) diradical of saturated- or unsaturated monocyclic group with one or more nitrogen atoms), which is obtained after removal of one hydrogen atom from said monocyclic group, or
(6) N(R³)-L- (wherein R³ is hydrogen or lower alkyl, and L is lower alkylene or lower alkenylene), and
R²¹, R²², R²³ or R²⁴ is
(1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
(2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl or
(3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.,
provided that
when A and two adjacent carbon atoms of the six membered ring to be bonded with A form benzene ring,
then 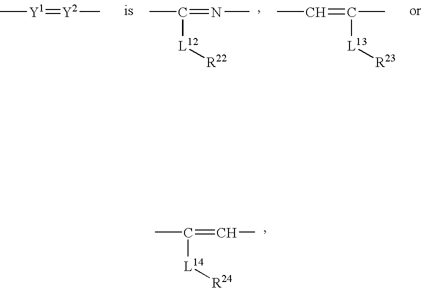
or its prodrug, or their salts.

11. A use of a compound of the formula (I): 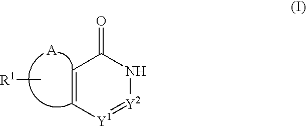
Wherein.
R¹ is hydrogen, halogen, lower alkyl or lower alkoxy,
A and two adjacent carbon atoms of the six membered ring to be bonded with A
form benzene ring, pyridine ring, or five to seven membered partially of saturated ring optionally containing one or more heteroatom(s) selected from the group consisting of nitrogen atom, oxygen atom, and sulfur atom, 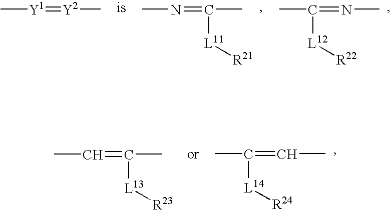
[wherein L¹¹, L¹², L¹³ and L¹⁴ is
(1) lower alkylene,
(2) lower alkenylene,
(3) cyclo(lower)alkylene,
(4) cyclo(lower)alkenylene,
(5) diradical of saturated- or unsaturated monocyclic group with one or more nitrogen atom(s), which is obtained after removal of one hydrogen atom from said monocyclic group, or
(6) N(R³)-L- (wherein R³ is hydrogen or lower alkyl, and L is lower alkylene and lower alkenylene), and
R²¹, R²², R²³ or R²⁴ is
(1) cyclic amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl,
(2) carbocyclic group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl, or
(3) amino group, which is substituted with phenyl optionally substituted with one or more suitable substituent(s) selected from the group consisting of halogen, nitro, lower alkoxy, lower alkyl, halo(lower)alkyl, halo(lower)alkoxy and phenyl, and which is optionally substituted with lower alkyl.,
provided that
when A and two adjacent carbon atoms of the six membered ring to be bonded with A form benzene ring,
then 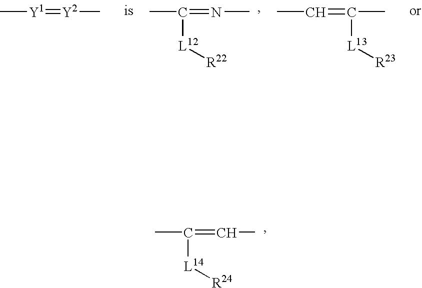
or its prodrug, or their pharmaceutically acceptable salts, for manufacturing a medicament for inhibiting PARP activity.
